(19) United States
(12) Patent Application Publication
(10) Pub. No.: US 2024/0293344 A1
(43) Pub. Date: Sep. 5, 2024

(54) Unsaturated Hydroxamic Acid Derivatives and Their Use for the Treatment and Prevention of an Ammonia-Associated Disease or Disorder

(71) Applicant: Versantis AG, Switzerland (CH)

(72) Inventors: Jean-Christophe Leroux, Zurich (CH); Filip Ilievski, Skopje (MK); Diana Evstafeva, Zurich (CH)

(21) Appl. No.: 18/573,596

(22) PCT Filed: Jun. 29, 2022
(86) PCT No.: PCT/IB2022/056066

§ 371 Date: Dec. 22, 2023

(30) Foreign Application Priority Data
Jun. 29, 2021 (EP) 21182587.2

(51) Int. Cl. A61K 31/16; A61K 9/00; A61K 9/08; A61K 9/48; A61K 47/40

(52) U.S. Cl. CPC A61K 31/16; A61K 9/0034; A61K 9/08; A61K 9/4891; A61K 47/40

(57) ABSTRACT

The present disclosure provides an enteral or urinary pharmaceutical formulation comprising (A) a compound of formula I, (I), wherein R₁ is a C1-C3 alkyl; X is —C(R₂)(R₃)—C(R₄)(R₅)— or —C≡C—, wherein R₂, R₃, R₄, and R₅ are each independently H or In methyl; Y is —CH═CH—CH₂—, —CH₂—CH═CH—; —C≡C—CH₂; —CH₂—C≡C—; or —C(R₇)(R₈)—C(R₉)(R₁₀)—C(R₁₁)(R₁₂)—, wherein R₇, R₈, R₉, R₁₀, R₁₁ and R₁₂, are each independently H or methyl; provided that (1) when X is —C≡C—, Y is —C(R₇)(R₈)—C(R₉)(R₁₀)—C(R₁₁)(R₁₂)— and that (2) when Y is not —C(R₇)(R₈)—C(R₉)(R₁₀)—C(R₁₁)(R₁₂)—, X is —C(R₂)(R₃)—C(R₄)(R₅)—; and R₆ is H or CH₃, or a stereoisomer or a mixture thereof, or a pharmaceutically acceptable salt, ester or solvate thereof; and (B) (a) at least one pharmaceutically acceptable excipient; or (b) at least one other therapeutic agent; or (c) a combination of (a) and (b); and a use thereof for the treatment or prevention of an ammonia-associated disease or disorder, or a symptom thereof.

### CROSS REFERENCE TO RELATED APPLICATIONS

This application is a PCT application Serial No 1B2022\* filed on Jun. 29, 2022 and published in English under PCT Article 21(2), which itself claims benefit of European application Serial No. 21182587.2, filed on Jun. 29, 2021. All documents above are incorporated herein in their entirety by reference.

### STATEMENT REGARDING FEDERALLY SPONSORED RESEARCH OR DEVELOPMENT

N.A.

### FIELD OF THE DISCLOSURE

The present disclosure relates to unsaturated hydroxamic acid (HA) derivatives and their use for the treatment and prevention of an ammonia-associated disease or disorder (e.g., hyperammonemia). More specifically, the present disclosure is concerned with the use of unsaturated HA derivatives to treat or prevent an ammonia-associated disease or disorder (e.g., hyperammonemia) in subjects suffering from e.g., hepatic encephalopathy (HE) or a urea cycle disorder (UCD).

### BACKGROUND OF THE DISCLOSURE

Urea cycle disorders (UCDs) and hepatic encephalopathy (HE) are diseases that are in part characterized by increased ammonia levels (Haberle, 2013). UCDs are caused by inherited deficiencies in the urea cycle enzymes and transporters such as ornithine transcarbamylase, carbamylphosphate synthetase 1, and others (Haberle, 2013; Walker, 2014). They can have serious health consequences including severe brain damage and hyperammonemic coma which is often fatal. HE is a complication of liver dysfunction which is characterized by a broad spectrum of neuropsychiatric abnormalities ranging from minor psychomotor impairment to coma (Swaminathan et al, 2018; Vilstrup et al, 2014). The manifestation of HE depends on the severity of liver dysfunction, presence of additional diseases (e.g., diabetes, kidney failure), patient age, degree of hyperammonemia, the severity of oxidative stress and inflammation, and other factors (Rose et al., 2020).

The pathogenesis of HE is a complex process where ammonia is considered to play a key role (Rose et al., 2020). Most ammonia in the human body is produced in the gut as a product of amino acid catabolism or hydrolysis of urea by urease-producing bacteria (Walker, 2014). In healthy individuals, ammonia is primarily detoxified through the urea cycle in hepatocytes and gets excreted as urea via kidneys. Also, other organs such as the brain, muscle, kidney are involved in the detoxification process by utilizing ammonia in the synthesis of glutamine. The main hypothesis of HE pathogenesis suggests that the dysfunctional liver is not able to eliminate ammonia efficiently leading to its accumulation in the systemic circulation. A condition with a blood ammonia level higher than 50 μmol/L is defined as hyperammonemia (Haberle, 2013). Ammonia is especially harmful to the brain where it is normally removed through the synthesis of glutamine in the astrocytes. However, excess ammonia induces the accumulation of glutamine in astrocytes, which disturbs brain homeostasis and leads to astrocytic swelling and brain edema. Additionally, ammonia is known to cause oxidative stress, mitochondrial dysfunction, disruption of cellular energy metabolism, and alterations in membrane potential both in neurons and astrocytes (Braissant et al., 2013; Bosoi et al., 2009).

Symptoms of HE are largely reversible when blood ammonia returns to normal levels (Braissant et al., 2013).

There is a need for an alternative or improved treatment of ammonia-associated disease or disorder (e.g., hyperammonemia).

The present description refers to a number of documents, the content of which is herein incorporated by reference in their entirety.

### SUMMARY OF THE DISCLOSURE

The present disclosure presents unsaturated derivatives of hydroxamic acids (HAs) for the treatment of an ammonia-associated disease or disorder, or a symptom thereof (e.g., HE and UCDs). These compounds have urease inhibitory activity. In particular, the efficacy of the compounds of the present disclosure was demonstrated by showing the decreased production of ammonia in rat's caecum content (Examples 6 and 10) as well as reduced blood ammonia levels in bile-duct ligated rats (model of hepatic encephalopathy (HE) associated with chronic liver cirrhosis) (Example 7) and in rats having N-Nitrosodiethylamine induced liver disease (model of acute liver disease) (Example 11) and was shown to be largely not cytotoxic (Example 8) and not mutagenic up to 1 mM (Example 9). Hydroxamates are generally known to exhibit mutagenic activity (Shen et al., 2016). The current hypothesis suggests that hydroxamic acids become mutagenic through a Lossen rearrangement. In neutral and basic conditions, the pre-formed 0-activated hydroxamic acid derivative loses a proton of the amide group and rapidly transforms into corresponding isocyanate reacting with DNA (Shen. et al. 2016). Data presented herein further suggests that 2-octynoHA in buffered solutions having a near neutral pH loses its hydroxamate group and converts into 5-pentylisoxazol-3-ol (Example 12). This conversion would reduce the absorption of intact 2-octynoHA and systemic exposure to the hydroxamate, thereby decreasing the mutagenic risk associated with this group. The oral bioavailability of 2-octynoHA was very low after the oral administration of an uncoated capsule (Example 13) and close to 0% after delivery via a colonic capsule (Example 13). The proposed delivery system would additionally ensure low systemic exposure and thus reduce possible side effects that would be associated to the mutagenic and cytotoxic potential of 2-octynoHA.

More specifically, in accordance with the present disclosure, there are provided the following items:
1. Enteral or urinary, preferably enteral, pharmaceutical formulation comprising (A) a compound of formula I,

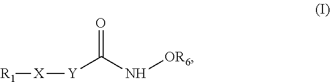

wherein R₁ is a C1-C3 alkyl;
X is —C(R₂)(R₃)—C(R₄)(R₅)— or —C≡C—,
   wherein R₂, R₃, R₄, and R₅ are each independently H or methyl;
Y is —CH═CH—CH₂—, —CH₂—CH═CH—; —C≡C—CH₂; —CH₂—C≡C—; or —C(R₇)(R₈)—C(R₉)(R₁₀)—C(R₁₁)(R₁₂)—,
   wherein R₇, R₈, R₉, R₁₀, R₁₁ and R₁₂, are each independently H or methyl;
provided that (1) when X is —C≡C—, Y is —C(R₇)(R₈)—C(R₉)(R₁₀)—C(R₁₁)(R₁₂)— and that (2) when Y is not —C(R₇)(R₈)—C(R₉)(R₁₀)—C(R₁₁)(R₁₂)—, X is —C(R₂)(R₃)—C(R₄)(R₅)—; and
R₆ is H or CH₃,
or a stereoisomer or a mixture thereof, or a pharmaceutically acceptable salt, ester or solvate thereof; and
(B) (a) at least one pharmaceutically acceptable excipient; (b) at least one other therapeutic agent; or (c) a combination of (a) and (b). Preferably the formulation comprising an enteric coating or being a sterile liquid formulation.
2. The formulation of item 1, wherein R₁ is ethyl.
3. The formulation of item 1, wherein
   (i) X is —C(R₂)(R₃)—C(R₄)(R₅)—;
   (ii) R₁ is ethyl;
   (iii) R₂ is H;
   (iv) R₃ is H;
   (v) R₄ is H;
   (vi) R₅ is H;
   (vii) R₆ is H;
   (viii) Y is —CH═CH—CH₂—, —CH₂—CH═CH—, —C≡C—CH₂—, or —CH₂—C≡C—; or
   (ix) a combination of at least two of (i) to (viii). Preferably at least, 3, 4, 5, 6, 7 or all 8 (i) to (viii).
4. The formulation of item 1, wherein
   (i) X is —C(R₂)(R₃)—C(R₄)(R₅)—;
   (ii) R₁ is ethyl;
   (iii) R₂ is H;
   (iv) R₃ is H;
   (v) R₄ is H;
   (vi) R₅ is H;
   (vii) R₆ is H; and
   (viii) Y is —CH═CH—CH₂—, —CH₂—CH═CH—, —C≡C—CH₂—, or —CH₂—C≡C—. Preferably Y being —CH₂—C≡C—, —CH═CH—CH₂—, or —CH₂—CH═CH—, more preferably Y being CH₂—C≡C— or —CH₂—CH═CH—, and most preferably Y being CH₂—C≡C—.
5. The formulation of any one of items 1 to 4, wherein the compound is of formula Ia:

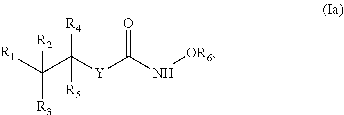

wherein R₁, R₂, R₃, R₄, R₅ and R₆ are as defined in any one of items 1 to 4 and Y is as defined in item 3 or 4, or a stereoisomer or a mixture thereof, or a pharmaceutically acceptable salt, ester or solvate thereof.
6. The formulation of any one of items 1 to 5, wherein Y is CH═CH—CH₂—, —CH₂—CH═CH— or —CH₂—C≡C—, preferably Y is CH═CH—CH₂—, or —CH₂—C≡C—. Preferably, R₁ is ethyl and/or one or more of R₂ to R₅ is H, or more preferably R₂ to R₅ are each H, and eventually R₁ is ethyl. Most preferably R₆ is H.
7. The formulation of any one of items 1 to 5, wherein Y is —CH₂—C≡C—. Preferably, R₁ is ethyl and/or one or more of R₂ to R₅ is H, or more preferably R₂ to R₅ are each H, and eventually R₁ is ethyl. Most preferably R₆ is H.
8. The formulation of item 1, wherein the compound is:

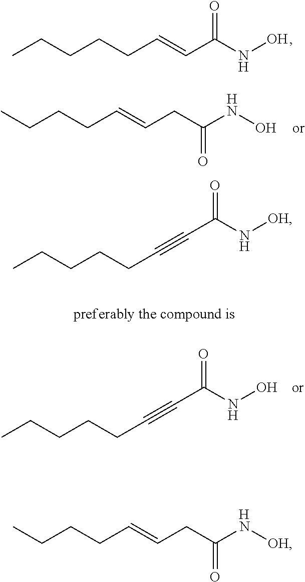

or a stereoisomer or a mixture thereof, or a pharmaceutically acceptable salt, ester or solvate thereof.
9. The formulation of item 1, wherein the compound is

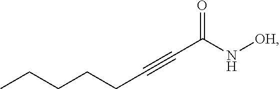

or a stereoisomer or a mixture thereof, or a pharmaceutically acceptable salt, ester or solvate thereof.
10. The formulation of any one of items 1 to 9, for enteral or urinary administration, preferably enteral administration, through a delayed release formulation.
11. The formulation of item 10, for delivery in the ileum or colon.
12. The formulation of any one of items 1 to 11, comprising an enteric coating or the formulation is a sterile liquid formulation, preferably the formulation comprises an enteric coating. In specific embodiments, the formulation further comprises an acid such as citric acid, tartaric acid, fumaric acid, maleic acid, succinic acid or ascorbic acid or any combination thereof (e.g., in a colonic formulation).
13. The compound, stereoisomer, mixture, salt, ester, solvate or formulation as defined in any one of items 1 to 12, for use in the treatment or prevention of an ammonia-associated disease or disorder, or a symptom thereof in a subject in need thereof.
14. The compound, stereoisomer, mixture, salt, ester, solvate or formulation for use of item 13, wherein the ammonia-associated disease or disorder is hyperammonemia, and the subject in need thereof preferably has hepatic encephalopathy or a urea cycle disorder.
15. The compound, stereoisomer, mixture, salt, ester, solvate or formulation for use of item 13 or 14, for enteral or urinary administration, preferably enteral administration and most preferably wherein the enteral administration is administration to the ileum or the colon.
16. Use of the compound, stereoisomer, mixture, salt, ester, solvate or formulation as defined in any one of items 1 to 12, for the treatment or prevention of an ammonia-associated disease or disorder, or a symptom thereof in a subject in need thereof.
17. Use of the compound, stereoisomer, mixture, salt, ester, solvate or formulation as defined in any one of items 1 to 12, in the manufacture of a medicament for the treatment or prevention of an ammonia-associated disease or disorder, or a symptom thereof in a subject in need thereof.
18. The use of item 16 or 17, wherein the ammonia-associated disease or disorder is hyperammonemia, and the subject in need thereof preferably has hepatic encephalopathy or a urea cycle disorder.
19. The use of item 16 or 17, wherein the compound, stereoisomer, mixture, salt, ester, solvate or formulation is for enteral administration, preferably wherein the enteral administration is delivery to the ileum or the colon.
20. Method of treating or preventing an ammonia-associated disease or disorder, or a symptom thereof in a subject in need thereof comprising administering a therapeutically effective amount of the compound, stereoisomer, mixture, salt, ester, solvate or formulation as defined in any one of items 1 to 12, to the subject.
21. The method of item 20, wherein the ammonia-associated disease or disorder is hyperammonemia, and the subject in need thereof preferably has hepatic encephalopathy or a urea cycle disorder.
22. The method of item 20, wherein the compound, stereoisomer, mixture, salt, ester, solvate or formulation is for administered enterally, preferably to the ileum or the colon.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-B: Comparison of urease inhibitory activity of acetohydroxamic acid (AHA) and octanohydroxamic acid (OHA) (FIG. 1A) and urease inhibitory activity of 2-octynohydroxamic acid (2-octynoHA) (FIG. 1B). Mean±SD (N=3−6).

FIG. 2: Effect of AHA, OHA and 2-octynoHA on viability of Caco-2 cells. Statistical significance was calculated by two-way analysis of variance (ANOVA) with Tukey's comparison test with * p<0.05, ** p<0.01, *** p<0.001. Mean±SD (N=6).

FIGS. 3A-E: Mutagenicity evaluation of 2-octynoHA in S. typhimurium strains (TA98 (FIG. 3A), TA100 (FIG. 3B), TA1535 (FIG. 3C), TA1537 (FIG. 3D)) and in the mixture of E. coli strains (wp2 [pKM101] and wp2 uvrA (FIG. 3E)) in the presence (+S9) or absence (−S9) of metabolic activation. The baseline is obtained by adding one standard deviation to the mean number of positive wells of the solvent control. As indicated by the dash-dotted line (−S9) and dashed line (+S9), test samples with the number of revertants more than two-fold induction over the baseline are considered to be mutagenic and they are marked with asterisk. PC stands for positive control. Mean±SD (N=3).

FIGS. 4A-E: Mutagenicity evaluation of OHA in S. typhimurium strains (TA98 (FIG. 4A), TA100 (FIG. 4B), TA1535 (FIG. 4C), TA1537 (FIG. 4D)) and in the mixture of E. coli strains (wp2 [pKM101] and wp2 uvrA (FIG. 4E)) in the presence (+S9) or absence (−S9) of metabolic activation. The baseline is obtained by adding one standard deviation to the mean number of positive wells of the solvent control. As indicated by the dash-dotted line (−S9) and dashed line (+S9), test samples with the number of revertants more than two-fold induction over the baseline are considered to be mutagenic and they are marked with asterisk. PC stands for positive control. Mean±SD (N=3).

FIGS. 5A-E: Mutagenicity evaluation of AHA in S. typhimurium strains (TA98 (FIG. 5A), TA100 (FIG. 5B), TA1535 (FIG. 5C), TA1537 (FIG. 5D)) and in the mixture of E. coli strains (wp2 [pKM101] and wp2 uvrA (FIG. 5E)) in the presence (+S9) or absence (−S9) of metabolic activation. The baseline is obtained by adding one standard deviation to the mean number of positive wells of the solvent control. As indicated by the dash-dotted line (−S9) and dashed line (+S9), test samples with the number of revertants more than two-fold induction over the baseline are considered to be mutagenic and they are marked with asterisk. PC stands for positive control. Mean±SD (N=3).

FIG. 6: Comparison of urease inhibitory activities of unsaturated alkylated HA derivatives consisting of 8 atoms of carbons. Mean±SD (N=3).

FIG. 7: In vivo efficacy of 2-octynoHA in N-Nitrosodiethylamine induced liver disease in rats. Mean±SD (N=10). Statistical significance was calculated by two-way analysis of variance (ANOVA) with Tukey's multiple comparisons test with *p<0.05.

FIGS. 8A-C: UHPLC-UV chromatograms of a solution of (FIG. 8A) 2-octynoHA 0.5 mg/mL in ultra-pure water; (FIG. 8B) 2-octynoHA 0.5 mg/mL in HBSS pH 7.4 supplemented with HEPES 15 mM (FIG. 8B); and of (FIG. 8C) 2-octynoHA 10 mM in KH₂PO₄ 200 mM pH 6.8 after overnight incubation at 37° C.

FIG. 9. Concentration profile of 2-octynoHA in dog plasma after I.V. administration (PK 1). Mean±SD (n=3).

FIG. 10. Concentration profile of 2-octynoHA in dog plasma following oral administration in an uncoated gelatin capsule (PK 2). Mean±SD (n=3).

### DESCRIPTION OF ILLUSTRATIVE EMBODIMENTS

The use of the terms “a” and “an” and “the” and similar referents in the context of describing the disclosure (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

### Compound

Compounds of the present disclosure are unsaturated derivatives of hydroxamic acid (HA) that have bacterial urease inhibitory activity. The mechanism of action of HAs is based on their ability to coordinate nickel atoms in the active site of urease and thereby prevent hydrolysis of urea to ammonia and carbamate (Muri and Barros, 2013). In specific embodiments, they have a higher bacterial urease inhibitory activity (and/or a lower IC₅₀ against bacterial urease activity) than that of acetohydroxamic acid (AHA) (IUPAC: N-hydroxyacetamide, CAS Registry Number: 546-88-3) and/or octanohydroxamic acid (OHA) (IUPAC: N-hydroxyoctanamide, CAS Registry Number: 7377-03-9). In other specific embodiments, compounds of the present disclosure have a lower cytotoxicity e.g., against Caco-2 cells than OHA. Cytotoxicity can be measured by e.g., Caco-2 cells viability in the presence of the compound of the present disclosure vs. in the presence of e.g., OHA.

Without being so limited, compounds of the present disclosure are of formula I or Ia:

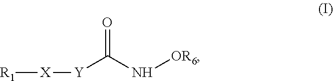

wherein R₁ is a C1-C3 alkyl;
X is —C(R₂)(R₃)—C(R₄)(R₅)— or —C≡C—,
   wherein R₂, R₃, R₄, and R₅ are each independently H or methyl;
Y is —CH═CH—CH₂—, —CH₂—CH═CH—; —C≡C—CH₂; —CH₂—C≡C—; or —C(R₇)(R₈)—C(R₉)(R₁₀)—C(R₁₁)(R₁₂)—,
   wherein R₇, R₈, R₉, R₁₀, R₁₁ and R₁₂, are each independently H or methyl;
provided that (1) when X is —C≡C—, Y is —C(R₇)(R₈)—C(R₉)(R₁₀)—C(R₁₁)(R₁₂)— and that (2) when Y is not —C(R₇)(R₈)—C(R₉)(R₁₀)—C(R₁₁)(R₁₂)—, X is —C(R₂)(R₃)—C(R₄)(R₅)—; and
R₆ is H or CH₃,
or a stereoisomer or a mixture thereof, or a pharmaceutically acceptable salt, ester or solvate thereof.

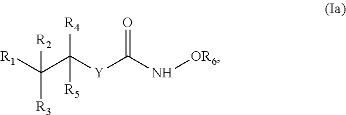

wherein R₁, R₂, R₃, R₄, R₅ and R₆ are as defined above and Y is as defined above, or a stereoisomer or a mixture thereof, or a pharmaceutically acceptable salt, ester or solvate thereof.

As used herein the term “C1-C3 alkyl” refer to methyl, ethyl or propyl.

Without being so limited, compounds of the present disclosure include:

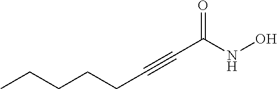

2-octynohydroxamic acid (2-octynoHA) N/IUPAC: N-hydroxyoct-2-ynamide CAS Registry Number: 85594-26-9

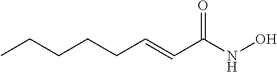

trans-2-octenohydroxamic acid (2-octenoHA) IUPAC: (E)-N-hydroxyoct-2-enamide CAS Registry Number: I3478448-8

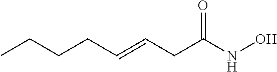

trans-3-octenohydroxamic acid (3-octynoHA) IUPAC: (E)-N-hydroxyoct-3-enamide

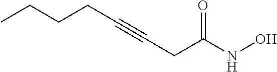

3-octynohydroxamic acid (3-octynoHA) IUPAC: N-hydroxyoct-3-ynamide and

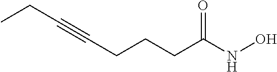

5-octynohydroxamic acid (5-octynoHA) IUPAC: N-hydroxyoct-5 ynamide

Also encompassed are stereoisomers, mixtures thereof, pharmaceutically acceptable salts, esters and solvates of the compounds of Formula I or Ia, preferably stereoisomers, mixtures thereof, pharmaceutically acceptable salts, and solvates thereof.

### Isomers, Tautomers and Polymorphs

As used herein, the term “isomers” refers to stereoisomers including diastereoisomers as well as the other known types of isomers.

Hydroxamic acids of the present disclosure exhibit Z/E isomerism (diastereoisomers) due to the rotation around C—N bond. The Z and E isomers co-exist in solution, the Z isomer displaying activity against urease. In addition, the present disclosure embraces all geometric isomers of the compounds of the disclosure. For example, in compounds of the disclosure incorporating a double or triple bond, both the cis- and trans-forms, as well as mixtures, are embraced within the scope of the disclosure.

Compounds of the present disclosure comprising a double bond may be in cis or trans configuration.

Within the present disclosure, it is to be understood that a compound of the disclosure may exhibit the phenomenon of tautomerism and that the formula drawings within this specification can represent only one of the possible tautomeric forms. It is to be understood that the disclosure encompasses any tautomeric form and is not to be limited merely to any one tautomeric form utilized within the formula drawings.

It is also to be understood that certain compounds of the disclosure may exhibit polymorphism, and that the present disclosure encompasses all such forms.

### Salts

The present disclosure relates to the compounds of the disclosure as hereinbefore defined as well as to salts thereof. The term “salt(s)”, as employed herein, denotes basic salts formed with inorganic and/or organic bases. Salts for use in pharmaceutical compositions/formulations will be pharmaceutically acceptable salts, but other salts may be useful in the production of the compounds of the disclosure. The term “pharmaceutically acceptable salts” refers to salts of compounds of the present disclosure that are pharmacologically acceptable and substantially non-toxic to the subject to which they are administered. More specifically, these salts retain the biological effectiveness and properties of the compounds of the disclosure and are formed from suitable non-toxic organic or inorganic acids or bases.

For example, where the compounds of the disclosure are sufficiently acidic, the salts of the disclosure include base salts formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and a cobalt salts; inorganic amine salts or substituted ammonium salts, such as e.g., trimethylammonium salts; and salts with organic bases (for example, organic amines) such as chloroprocaine salts, dibenzylamine salts, dicyclohexylamine salts, dicyclohexylamines, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts. Preferred salts include those formed with sodium, lithium, potassium, calcium and magnesium.

Such salts can be formed routinely by those skilled in the art using standard techniques. Indeed, the chemical modification of a pharmaceutical compound (i.e., drug) into a salt is a technique well known to pharmaceutical chemists, (See, e.g., H. Ansel et. al., Pharmaceutical Dosage Forms and Drug Delivery Systems (6th Ed. 1995) at pp. 196 and 1456-1457, incorporated herein by reference). Salts of the compounds of the disclosure may be formed, for example, by reacting a compound of the disclosure with an amount of base, such as an equivalent amount, in a medium such as one in which the salt precipitates or in an aqueous medium followed by drying.

### Esters

The present disclosure relates to the compounds of the disclosure as well as to the hydroxamate esters thereof. The term “ester(s)”, as employed herein, refers to compounds of the disclosure or salts thereof in which a hydroxy group has been converted to the corresponding esters using a carbonyl group-containing reagent and a coupling reagent. Such hydroxamate esters may be used as prodrugs. Esters for use in pharmaceutical compositions/formulations will be pharmaceutically acceptable esters, but other esters may be useful in the production of the compounds of the disclosure.

The term “pharmaceutically acceptable esters” refers to esters of the compounds of the present disclosure that are pharmacologically acceptable and substantially non-toxic to the subject to which they are administered. More specifically, these esters retain the biological effectiveness and properties of the compounds of the disclosure after hydrolysis, and can act as prodrugs which, when delivered to the gastrointestinal tract of a warm-blooded animal, cleave in such a manner as to produce the parent compounds.

Esters of the compounds of the present disclosure include hydroxamic acid esters obtained by esterification, in which the non-hydroxamic acid portion of the ester grouping is selected from straight or branched chain alkyl (for example, ethyl, n-propyl, t-butyl, n-butyl, methyl, propyl, isopropyl, butyl, isobutyl, or pentyl), n-hexyl, alkoxyalkyl (for example, methoxymethyl, acetoxymethyl, and 2,2-dimethylpropionyloxymethyl), aralkyl (for example, benzyl), aryloxyalkyl (for example, phenoxymethyl), aryl (for example, phenyl optionally substituted with, for example, halogen, C1-4 alkyl, or C1-4 alkoxy, or amino).

Esters of the compounds of the disclosure may form salts. Where this is the case, this is achieved by conventional techniques as described above.

### Solvates

The compounds of the disclosure may exist in unsolvated as well as solvated forms with solvents such as water, ethanol, and the like, and it is intended that the disclosure embrace both solvated and unsolvated forms.

“Solvate” means a physical association of a compounds of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Solvates for use in pharmaceutical compositions/formulations will be pharmaceutically acceptable solvates, but other solvates may be useful in the production of the compounds of the disclosure.

As used herein, the term “pharmaceutically acceptable solvates” means solvates of compounds of the present disclosure that are pharmacologically acceptable and substantially non-toxic to the subject to which they are administered. More specifically, these solvates retain the biological effectiveness and properties of the compounds of the disclosure and are formed from suitable non-toxic solvents.

Non-limiting examples of suitable solvates include ethanolates, methanolates, and the like, as well as hydrates, which are solvates wherein the solvent molecule is water.

Preparation of solvates is generally known. Thus, for example, Caira, 2004, incorporated herein by reference, describe the preparation of the solvates of the antifungal fluconazole in ethyl acetate as well as from water. Similar preparations of solvates, hemisolvate, hydrates and the like are described by van Tonder, 2004; Bingham, 2001, both incorporated herein by reference.

A typical, non-limiting, process for preparing a solvate involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example infrared spectroscopy, can be used to show the presence of the solvent (or water) in the crystals as a solvate (or hydrate).

As used herein the term “higher” in the context of a “higher bacterial urease inhibitory activity than that of AHA and/or OHA” refers to a bacterial urease inhibitory activity obtained with a compound of the present disclosure that is at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, 55%, 60%, 65%, 70%, 75%, 80%, 85%, 90%, 95%, 100%, 150%, 200%, 250%, 300%, 400%. 500%, 600%, 700%, 800%, 900% or 1000% higher etc. than the corresponding bacterial urease inhibitory activity obtained with AHA and/or OHA, respectively.

As used herein the term “lower” in the context of a “lower IC₅₀ against bacterial urease activity than that of AHA and/or OHA” or of a “lower cytotoxicity against e.g., Caco-2 cells than that of OHA” refers to the IC₅₀ of a compound of the present disclosure against bacterial urease activity or the cytotoxicity of a compound of the present disclosure against e.g., Caco-2 cells, respectively, that is at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900% or 1000% lower than the corresponding IC50 or cytotoxicity, respectively obtained with AHA and/or OHA, respectively.

In comparison to lactulose and rifaximin for example, compounds of the present disclosure (e.g., 2-octynoHA) specifically act on urease, do not bear the risk of inducing antibiotic resistance, and because of a high urease inhibitory activity, can be used in relatively low dose to achieve a significant reduction in ammonia concentration and consequently, may have lower risks to develop adverse effects than other anti-urease compounds. In comparison to existing HAs, (AHA, OHA) whose efficacy in reducing ammonia production was shown in early studies, the IC₅₀ of 2-octynoHA was found to be approximately 10 times lower in an in vitro assay (Example 6).

Route Compounds of the present disclosure may be administered through the gastrointestinal tract i.e., enterally (i.e., orally, or rectally/intracolonically) or through the urinary tract (e.g., via bladder instillation) for the prevention or treatment of an ammonia-associated disease or disorder, or a symptom thereof. For example, the compounds of the present disclosure can be formulated in a colonic formulation to allow their release in the distal intestinal segments thereby minimizing systemic absorption and further reducing the risk of producing side effects. When the ammonia-associated disease or disorder is a urinary tract infection, compound can be administered orally or through the urinary tract (e.g., bladder instillation).

### Compositions/Formulations

The present disclosure also relates to the use of the compounds in the preparation of a medicament (composition/formulation).

The present disclosure also relates to pharmaceutical compositions/formulations comprising the compounds and at least one pharmaceutically acceptable excipient.

The present disclosure relates to pharmaceutical formulation for enteral administration, namely through the oral or rectal/intracolonical route. The present disclosure relates to enteral pharmaceutical formulations.

The present disclosure also relates to pharmaceutical formulation for administration through the urinary tract (e.g., via bladder instillation).

When the oral route is used for example, the compounds described herein are oral formulations that can be in the form of solids e.g., tablets (coated or not), hard or soft capsules (coated or not), suppositories; or liquids e.g., solutions, suspensions, or emulsions.

When the rectal/intracolonic route is used for example, the compounds described herein are rectal or intracolonic formulations that can be in the form of e.g., suppositories or liquids (e.g., solutions, suspensions, or emulsions).

When the urinary route is used for example (e.g., via bladder instillation), the compounds described herein are urinary tract formulations that can be used in the form of e.g., sterile liquid (e.g., solution or suspension (comprising a majority of particles with particle diameters smaller than about 10 micrometers)).

Enteral formulations can be prepared to achieve controlled (e.g., delayed) release of the compounds of the present disclosure at specific locations of the gastrointestinal tract. For example, colonic delivery can be achieved with e.g., an enterically coated oral formulation.

For the preparation of liquid formulations, the compositions/formulations of the disclosure can contain at least one pharmaceutically acceptable liquid carrier including, without limitation, an aqueous or non-aqueous carrier. Examples of non-aqueous carriers include, without limitation, ethanol, propylene glycol, polyethylene glycol, triglycerides, etc. Examples of aqueous carriers include, without limitation, water, saline, etc.

The carrier may further comprise at least one additional excipient to produce e.g., a solution of cyclodextrins such as (2-hydroxypropyl)-beta-cyclodextrin (HPβCD) or a buffered solution. More particularly, the carrier may contain one or more of at least one excipient increasing the bioavailability, water solubility or stability of the compounds of the present disclosure. Without being so limited, such at least one excipient may comprise one or more of at least one solvent (e.g., DMSO); at least one dispersion aid such as a surfactant (e.g., polysorbate); at least one excipient for increasing aqueous solubility of compounds of the disclosure such as at least one saccharide (cyclodextrin such as (2-hydroxypropyl)-beta-cyclodextrin (HPβCD), saccharose, invert sugar and glucose); at least one stabilizing agent such as at least one pH modulator (e.g., at least one salt, at least one acid such as citric acid, tartaric acid, fumaric acid, maleic acid, succinic acid, and/or ascorbic acid); and/or at least one antioxidant (e.g., ascorbic acid). Urinary tract liquid formulations for example would be a sterile aqueous liquid (e.g., solution or suspension (comprising a majority of particles with particle diameters smaller than about 10 micrometers)) comprising one or more of a solvent (e.g., DMSO), surfactant (e.g., polysorbate) or cyclodextrin and eventually at least one pH modulator (e.g., salt, citric acid).

For the preparation of solid formulations (e.g., tablets (coated or not), capsules (coated or not), or suppositories), the compounds of the present disclosure may be admixed with any known pharmaceutically inert, inorganic or organic excipient and/or carrier. Examples of suitable excipients/carriers include one or more of lactose, at least one saccharide (cyclodextrin e.g., (2-hydroxypropyl)-beta-cyclodextrin (HPβCD), cellulose and its derivatives, saccharose, invert sugar and glucose, maize starch or derivatives thereof), talc or stearic acid or salts thereof, stabilizing agents at least one pH modulator (e.g., at least one salt, at least one acid such as citric acid, tartaric acid, fumaric acid, maleic acid, succinic acid, and ascorbic acid), and/or at least one antioxidant (e.g., ascorbic acid). Without being so limited, when the compounds of the disclosure are intended for delivery to the distal part of the intestine (such as the colon), coating the tablets or capsules with a coating degrading or dissolving in the ileum and/or colon such as methacrylic acid copolymers (e.g., Eudragit™ S100) or using a polymer matrix that is degraded in the ileum and/or colon. Alternative ways of achieving colonic delivery are described in Philip et al., 2010. In specific embodiments, the formulation is not a cream, a lotion, or an ointment.

In preferred embodiments, the chemical stability of compound of the present disclosure (e.g., 2-octynoHA) in compositions/formulations (e.g., enteric capsule, tablet) of the present disclosure is increased by combining it with an acid (i.e., citric acid, tartaric acid, fumaric acid, maleic acid, succinic acid, or ascorbic acid).

For the preparation of emulsions, the compounds of the present disclosure may be admixed with any known pharmaceutically inert, inorganic or organic excipient and/or carrier. Examples of suitable excipients/carriers include water, surfactants (e.g., polysorbates, sorbitan esters, sodium lauryl sulfate, etc.), oils (e.g., mineral or vegetable oils).

The compositions/formulations of the present disclosure may contain at least one of: antifrictional agents, disintegrants, preserving agents, stabilizing agents, wetting agents, sweeteners, colorants, odorants, salts, buffers, and antioxidants. They may also contain other therapeutically active agents.

It is a prerequisite that all excipients used in the manufacture of the compositions/formulations of the present disclosure are non-toxic and more generally pharmaceutically acceptable. As used herein, “pharmaceutically acceptable” such as pharmaceutically acceptable carrier, excipient, etc., means pharmacologically acceptable and substantially non-toxic to the subject to which the particular composition/formulation of the present disclosure is administered.

### Combination Therapy

Compounds of the present disclosure and or compositions/formulations thereof can also be administered in a combination therapy, i.e., combined with at least one other therapeutic agent or therapy for simultaneous or sequential administration. The combination therapy can include a compound or composition/formulation of the present disclosure combined with at least one other therapeutic agent or therapy. Such other therapeutic agent or therapy can be an agent or therapy for the prevention or treatment of an ammonia-associated disease or disorder or symptom thereof or for the prevention or treatment of another symptom of the underlying disease or condition.

For example, the combination therapy can include a compound or composition/formulation of the present disclosure combined with at least one other drug or therapy used for the prevention or treatment of the ammonia-associated disease or disorder; or with a drug or therapy used for the prevention or treatment of at least one other symptom of a disease or disorder of the subject having the ammonia-associated disease or disorder (underlying disease or disorder). In this context, examples of therapeutic agents or therapies that may be administered in combination (simultaneously or sequentially) with the compound or composition/formulation of the present disclosure include another compound or composition/formulation of the present disclosure and/or at least one other therapeutic agent or therapy. When used to treat hyperammonemia, the at least one other therapeutic agent or therapy can be at least one of non-absorbable disaccharides such as lactulose or lactilol, rifaximin, a branched-chain amino acid, neomycin, metronidazole, probiotic (such as but not limited to VSL #3 (Rivera-Flores 2020)), a glutaminase inhibitor, L-ornithine-L-aspartate, hemodialysis, peritoneal dialysis, sodium phenylbutyrate (e.g., Buphenyl®), sodium phenylacetate, sodium benzoate, a combination of sodium phenylacetate/sodium benzoate (e.g., Ammonul®, Ucephan®), glycerol phenylbutyrate (e.g., Ravicti®) or carglumic acid. When used to treat a urinary tract infection, the at least one other therapeutic agent can be an antibiotic such as trimethoprim/sulfamethoxazole (Bactrim, Septra, others), fosfomycin (Monurol), nitrofurantoin (Macrodantin, Macrobid), cephalexin (Keflex), ceftriaxone, a fluoroquinolone such as ciprofloxacin (Cipro), levofloxacin and others. When used to treat an ulcer, the at least one other therapeutic agent can be an antibiotic such as amoxicillin (Amoxil), clarithromycin (Biaxin), metronidazole (Flagyl), tinidazole (Tindamax), tetracycline and levofloxacin; a proton pump inhibitor such as omeprazole (Prilosec), lansoprazole (Prevacid), rabeprazole (Aciphex), esomeprazole (Nexium) and pantoprazole (Protonix); an acid blocker such as famotidine (Pepcid AC), cimetidine (Tagamet HB) and nizatidine (Axid AR), an antacid that neutralize stomach acid; and/or cytoprotective agents such as sucralfate (Carafate) and misoprostol (Cytotec).

When used in such combination, the compounds or compositions/formulations of the present disclosure could enable the administration of a lower dose of the other drug or therapy (e.g., anti-hyperammonemia drug such as lactulose) and thereby reduce the side effects associated with such drug or therapy, such as diarrhea, nausea, bloating, and flatulence.

### Method of Prevention or Treatment

The present disclosure is drawn to a compound, stereoisomer, mixture, salt, ester or solvate or formulation of the present disclosure for use in the treatment or prevention of an ammonia-associated disease or disorder, or a symptom thereof in a subject in need thereof. In a specific embodiment, the ammonia-associated disease or disorder is hyperammonemia. In another specific embodiment, the subject in need thereof has hepatic encephalopathy or a urea cycle disorder.

As used herein an “ammonia-associated disease or disorder, or a symptom thereof” includes pathologically high levels of ammonia in bodily fluids and tissues such as blood (i.e., hyperammonemia), urinary tract (e.g., bladder) and stomach, and underlying diseases or conditions that are associated therewith. In specific embodiments “ammonia-associated disease or disorder” refers to hyperammonemia (induced by e.g., impaired liver function, drug-induced hyperammonemia, inborn deficiency in hepatic ammonia metabolism (primary hyperammonemia), inborn deficiency affecting intermediary hepatic ammonia metabolism (secondary hyperammonemia), and underlying diseases or disorders that are associated to hyperammonemia, including but not limited to hepatic encephalopathy (HE), liver cirrhosis, acute liver failure, acute-on-chronic liver failure, portosystemic bypass/shunting, and urea cycle disorders (UCDs), or a symptom thereof. In other specific embodiments “ammonia-associated disease or disorder” refers to urinary tract infections caused by e.g., Proteus, Klebsiella, Pseudomonas and/or Staphylococcus species (i.e., urea-splitting urinary tract infections). They display pathologically high levels of ammonia in the urinary tract. In other specific embodiments, “ammonia-associated disease or disorder” refers to ulcers associated with Helicobacter Pylori infections. They display pathologically high levels of ammonia in the stomach.

As used herein in relation to an ammonia-associated disease or disorder, the term “a symptom thereof” include any symptom of the foregoing diseases and disorders. For example, when the disease or disorder is hyperammonemia, symptoms include cognitive deterioration.

UCDs are caused by inherited deficiencies in the urea cycle enzymes and transporters such as ornithine transcarbamylase (OTC), argininosuccinate synthetase (citrullinemia type 1) (ASS), arginase 1 (ARG1), argininosuccinate lyase (argininosuccinic aciduria) (ASL), carbamoylphosphate synthetase 1 (CPS1), and N-acetylglutamate synthase (NAGS). UCDs are named based on the initials of the missing or defective enzyme.

As used herein, the term “prevent/preventing/prevention” or “treat/treating/treatment”, refers to eliciting the desired biological response, i.e., a prophylactic and therapeutic effect, respectively in a subject. In accordance with the present disclosure, the therapeutic effect comprises one or more of a decrease/reduction in the severity, intensity and/or duration of high levels of ammonia (e.g., hyperammonemia) or a symptom thereof following administration of the compound (or composition/formulation) of the present disclosure when compared to its severity, intensity and/or duration in the subject prior to treatment or as compared to that/those in a non-treated control subject having high levels of ammonia (e.g., hyperammonemia) or a symptom thereof. In accordance with the disclosure, a prophylactic effect may comprise a delay in the onset of the ammonia-associated disease or disorder, or a symptom thereof in an asymptomatic subject at risk of experiencing the ammonia-associated disease or disorder, or a symptom thereof at a future time; or a decrease/reduction in the severity, intensity and/or duration of ammonia-associated disease or disorder, or a symptom thereof occurring following administration of the compound (or composition/formulation) of the present disclosure, when compared to the timing of their onset or their severity, intensity and/or duration in a non-treated control subject (i.e. asymptomatic subject at risk of experiencing the ammonia-associated disease or disorder), or a symptom thereof; and/or a decrease/reduction in the progression of any pre-existing ammonia-associated disease or disorder, or a symptom thereof in a subject following administration of the compound (or composition/formulation) of the present disclosure when compared to the progression of an ammonia-associated disease or disorder, or a symptom thereof in a non-treated control subject having such pre-existing ammonia-associated disease or disorder, or a symptom thereof. As used herein, in a therapeutic treatment, the compound (or composition/formulation) of the present disclosure is administered after the onset of the ammonia-associated disease or disorder, or a symptom thereof. As used herein, in a prophylactic treatment, the compound (or composition/formulation) of the present disclosure is administered before the ammonia-associated disease or disorder, or a symptom thereof or after the onset thereof but before the progression thereof.

A “therapeutically effective amount” or “effective amount” or “therapeutically effective dosage” of a specific compound (or composition/formulation thereof) of the disclosure can result in an inhibition of urease activity in a subject.

As used herein the term “subject” refers to an animal such as a mammal. In a specific embodiment, it refers to a human. It also refers to pets or other animals (e.g., pets such as cats, dogs, horses, etc.; and cattle, swine, poultry, etc.).

As used herein the terms “subject in need thereof” refer to a subject who would benefit from receiving an effective amount of the compound or composition/formulation of the present disclosure. In a specific embodiment, the subject suffers from an ammonia-associated disease or disorder, or a symptom thereof. In specific embodiment, the subject suffers from HE or a UCD.

### Kits

Also within the scope of the disclosure are kits comprising at least one type of compound or composition/formulation of the present disclosure and instructions for their use e.g., for the prevention or treatment of an ammonia-associated disease or disorder, or a symptom thereof. The kit can further contain at least one other agent for the prevention or treatment of the ammonia-associated disease or disorder or symptom thereof or for the prevention or treatment of another symptom of the underlying disease or disorder, or one or more additional compounds of the disclosure. Kits typically include a label indicating the intended use of the contents of the kit. The term label includes any writing, or recorded material supplied on or with the kit, or which otherwise accompanies the kit. The kit may further comprise one or more container(s), reagent(s), administration device(s).

### Dosage

The dosages in which the compounds of the disclosure or compositions/formulations thereof are administered will depend on many factors including the age, other medications taken by subject (e.g., for other diseases or conditions) and other clinically relevant factors. Typically, the amount of the compounds of the disclosure or compositions/formulations thereof contained within a single dose will be an amount that effectively treats the ammonia-associated disease or disorder or symptom thereof (e.g., hyperammonemia, HE or UCDs) without inducing significant toxicity.

The effective amount of the compounds of the disclosure or compositions/formulations thereof may also be measured directly. The effective amount may be given daily or weekly or fractions thereof. Typically, the dose of compounds of the disclosure ranges from about 1 mg up to about 500 mg per kg of body weight per day (e.g., 1 mg, 10 mg, 50 mg, 100 mg, or 250 mg/kg of body weight per day). Dosages may be provided in either a single or multiple dosage regimen. For example, in some embodiments the effective amount may range from about 250 mg to about 500 mg per day, from about 500 mg to about 1000 mg per day, about 1 gram per day, about 2-12 grams per day, about 14 g to about 86 grams of the composition/formulation per week, etc.

### Screening Methods

In accordance with another aspect of the present disclosure, there is provided a method of identifying a compound for the prevention or treatment of an ammonia-associated disease or disorder, or a symptom thereof, said method comprising contacting a bacterial urease (or a cell expressing same) with a candidate compound and determining the effect of said candidate compound on the bacterial urease activity (e.g., conversion of urea into ammonia), wherein a decrease in the activity of the urease in the presence as compared to in the absence of said candidate compound is an indication that said candidate compound may prevent or treat an ammonia-associated disease or disorder, or a symptom thereof, in e.g., HE and/or UCDs or a symptom thereof.

Other objects, advantages and features of the present disclosure will become more apparent upon reading of the following non-restrictive description of specific embodiments thereof, given by way of example only with reference to the accompanying drawings.

The present disclosure is illustrated in further details by the following non-limiting examples.

### Example 1: Synthesis of 2-Octynohydroxamic Acid

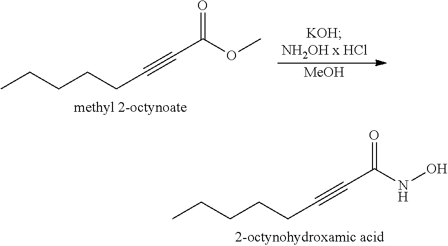

KOH (742.2 mg, 13 mmol) and hydroxylamine hydrochloride (900 mg, 13 mmol) were dissolved in 3 and 6 mL of methanol (MeOH), respectively. The KOH solution was added to the hydroxylamine hydrochloride solution with stirring under inert atmosphere on ice, whereupon a white precipitate (KCl) was observed. Once all the KOH had been added, the obtained mixture was allowed to stir for 20 min to ensure complete precipitation of KCl. The mixture was filtered under vacuum, and methyl 2-octynoate (1 g, 6.5 mmol) was added to the filtrate. The reaction mixture was stirred at room temperature for 24 h, then quenched with 20 mL dichloromethane (DCM) and washed with brine (20 mL). The crude was mixed with celite, loaded in a solid load cartridge and then purified by CombiFlash™ medium pressure liquid chromatography (MPLC) system (DCM/MeOH) to give 39 mg of white pinkish 2-octynohydroxamic acid (2-octynoHA) (yield: 4%). ¹H NMR and ¹³C NMR spectra were recorded for 2-octynoHA dissolved in deuterated dimethyl sulfoxide (DMSO-d6).

¹H NMR (400 MHz, DMSO-d6) δ 10.97 (s, 1H), 9.09 (s, 1H), 2.33 (t, J=8.0 Hz, 2H), 1.52-1.45 (m, 2H), 1.40-1.22 (m, 4H), 0.88 (t, J=8.0 Hz, 3H).

¹³C NMR (100 MHz, DMSO-d6) δ 150.84, 88.32, 74.39, 30.82, 27.49, 22.05, 18.17, 14.28.

### Example 2: Synthesis of Trans-2-Octenohydroxamic Acid

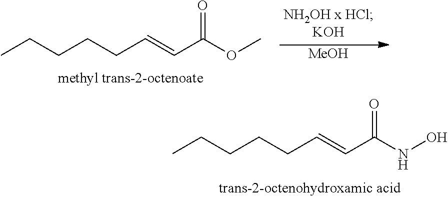

Two separated solutions of hydroxylamine hydrochloride (1.33 g, 19.2 mmol) and KOH (2.15 g, 38.4 mmol) were prepared in MeOH and placed on ice. The solution of KOH was added to the hydroxylamine hydrochloride solution, the obtained mixture was allowed to stir for 20 min. Then, the solution of methyl trans-2-octenoate (0.3 g, 1.92 mmol) in MeOH was added to the mixture, which was then stirred on ice for 5 min, removed and stirred again at room temperature for 24 h. The solution was acidified with 2M HCl aq, extracted with DCM (3×50 mL) and dried over MgSO₄. The product was purified by CombiFlash™ MPLC system (DCM/MeOH) and gave 99.6 mg of trans-2-octenohydroxamic acid (2-octenoHA) (yield: 33%). ¹H-NMR and ¹³C NMR spectra were recorded for 2-octenoHA dissolved in DMSO-d6.

¹H NMR (400 MHz, DMSO-d6) δ 10.51 (s, 1H), 8.83 (s, 1H), 6.63 (m, 1H), 5.73 (d, J=16 Hz, 1H), 2.12 (m, 2H), 1.46-1.19 (m, 6H), 0.87 (t, J=8.0 Hz, 3H). ¹³C NMR (100 MHz, DMSO-d6) δ 163.18, 142.70, 121.70, 31.69, 31.22, 27.96, 22.36, 14.34.

### Example 3: Synthesis of Trans-3-Octenohydroxamic Acid

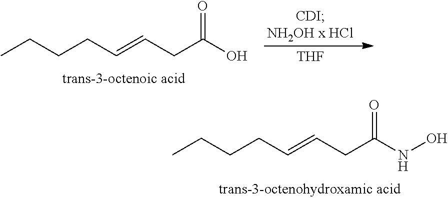

1,1′-Carbonyldiimidazole (CDI) (855 mg, 5.27 mmol) was added to a solution of trans-3-octenoic acid (500 mg, 3.52 mmol) in 5 mL dry tetrahydrofuran (THF) and stirred for 1 h. Then, hydroxylamine hydrochloride (500 mg, 7.25 mmol) was added to the mixture and left to stir overnight. The reaction was quenched with 5% aq. KHSO₄ (30 ml) and extracted with dichloromethane (2×30 ml). The combined organic phases were washed with brine (30 ml) and dried over Na₂SO₄. The extract was filtered, concentrated in vacuo and purified using the CombiFlash™ MPLC system to give 205.22 mg of trans-3-octenohydroxamic acid (3-octenoHA) as yellowish crystal (yield: 37%). ¹H-NMR and ¹³C NMR spectra were recorded for 3-octenoHA dissolved in DMSO-d6.

¹H NMR (400 MHz, DMSO-d6) δ 10.37 (s, 1H), 8.70 (s, 1H), 5.56-5.36 (m, 2H), 2.67 (d, J=4 Hz, 2H), 1.94-1.99 (m, 2H), 1.34-1.21 (m, 4H), 0.90-0.82 (t, J=8.0 Hz, 3H).

¹³C NMR (100 MHz, DMSO-d6) δ 167.84, 133.40, 124.12, 36.96, 32.00, 31.36, 22.07, 14.25.

### Example 4: Synthesis of 3-Octynohydroxamic Acid

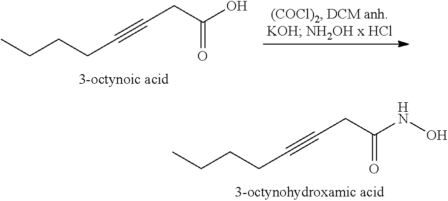

Oxalyl chloride (COCl)₂ (0.3 mL, 3.57 mmol) was added to a solution of 3-octynoic acid (204.6 mg, 1.43 mmol) in 4.2 mL dry DCM. The reaction was stirred at room temperature for 1 h. The solvent was evaporated under reduced pressure. Solutions of hydroxylamine hydrochloride (205 mg, 2.86 mmol) in 3 mL MeOH, KOH (208 mg, 2.86 mmol) in 3 mL of MeOH and 200 μL hydroxylamine (50% solution in water) were added to the evaporated mixture. The reaction was stirred at room temperature for 1 h, whereupon a white precipitate was observed. Then, the reaction mixture was acidified with 0.1 M HCl and extracted with DCM (3×10 mL). The combined organic phases were washed with brine, dried with MgSO₄ and filtered. The solvent was evaporated, and the residue was purified using the CombiFlash™ MPLC system to give 29.7 mg of 3-octynohydroxamic acid (3-octynoHA) as orange oil (yield: 13.4%). ¹H-NMR and ¹³C NMR spectra were recorded for 3-octynoHA dissolved in DMSO-d6.

¹H NMR (400 MHz, DMSO-d6) δ 10.48 (s, 1H), 8.89 (s, 1H), 2.92 (t, J=4 Hz, 2H), 2.17-2.13 (m, 2H), 1.46-1.29 (m, 4H), 0.87 (t, J=8.0 Hz, 3H).

¹³C NMR (100 MHz, DMSO-d6) δ 164.41, 82.60, 74.49, 30.81, 24.49, 21.80, 18.21, 13.93.

### Example 5: Synthesis of 7-Octynohydroxamic Acid

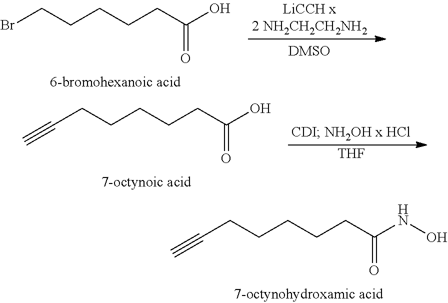

7-octynohydroxamic acid (7-octynoHA) was synthesized from 7-octynoic acid which was produced via the SN₂ type alkylation reaction using a Li-acetylide and 6-bromohexanoic acid. In a flame dried flask, lithium acetylide diamine complex (245.85 mg, 7.69 mmol) was dissolved in anhydrous DMSO and cooled to 0° C. Then, 6-bromohexanoic acid (1 g, 5.13 mmol) was added, and the reaction mixture was left to stir for 3 h. The reaction was quenched on ice with brine, acidified with 2M HCl, and extracted with DCM (3×50 ml). The combined organic phases were dried over MgSO₄. The solvent was evaporated to obtain colorless oil of 7-octynoic acid which was used in the next reaction without further purification. 7-octynoic acid (900 mg, 6.42 mmol) obtained in the previous step was dissolved in THF. Then, CDI (3.12 g, 19.26 mmol) was added to the solution, and the mixture was stirred vigorously for 1 h. After that, hydroxylamine hydrochloride (2.23 g, 32.1 mmol) was added, and the reaction was left to stir overnight at room temperature. After 20 h, the reaction was quenched with 30 ml of KHSO₄ and extracted with DCM (3×50 ml). The combined organic phases were dried over MgSO₄ and concentrated in vacuo to obtain yellowish oil. The crude was purified by column chromatography with DCM/MeOH (98/2-90/10, v/v). 7-octynoHA was obtained as white crystals (0.4 g, 40% yield). ¹H-NMR and ¹³C NMR spectra were recorded for 7-octynoHA dissolved in DMSO-d6.

¹H NMR (400 MHz, DMSO-d6) δ 10.33 (s, 1H), 8.66 (s, 1H), 2.74 (t, J=4 Hz, 1H), 2.12-2.16 (m, 2H), 1.94 (t, J=8.0 Hz, 2H), 1.40-1.53 (m, 4H), 1.37-1.28 (m, 2H).

¹³C NMR (100 MHz, DMSO-d6) δ 168.91, 84.40, 71.11, 54.83, 32.07, 27.61, 24.54, 17.53.

### Example 6: Activity of 2-Octynohydroxamic Acid in the Rat Caecum Content Assay

To evaluate the inhibitory activity of HAs, the inventors first established an in vitro assay using rat caecum content.

The use of caecum content samples for the initial screening of urease inhibitors allowed to rapidly identify stable and membrane permeable inhibitors that cover a broad spectrum of bacterial ureases present in the gastrointestinal tract.

According to the herein established protocol, rat caecum content provided by the ETH Phenomics center was diluted in 200 mM potassium phosphate monobasic buffer pH 6.8 and centrifuged at 100×g to remove large particles. The supernatant with dispersed bacteria was collected, mixed with urea and then incubated with an increasing range of inhibitor concentrations for 30 min at 37° C. with constant shaking. In this setup initial concentrations of bacteria and urea were adjusted to the values that lead to the production of ammonia at a final concentration of approximately 1000 μM. Bacterial urease converts urea to ammonia, which was quantified by an enzymatic assay (Ammonia assay, Randox Laboratories, UK).

The established assay was then used to evaluate the potency of commercially available HAs as well as synthesized HAs of the present disclosure. As most of the tested HAs have low aqueous solubility, cyclodextrins were used to prepare water-soluble formulations of the compounds. In this study, a stock solution of acetohydroxamic acid (AHA) was prepared in DMSO, and stock solutions of octanohydroxamic acid (OHA) and 2-octynohydroxamic acid (2-octynoHA) were prepared by dissolving them in aqueous solutions of 2-hydroxypropyl-p-cyclodextrin (HPβCD) in 1:2 and 1:4 molar ratios, respectively. The obtained mixtures were sonicated and heated at 37° C. until fully dissolved.

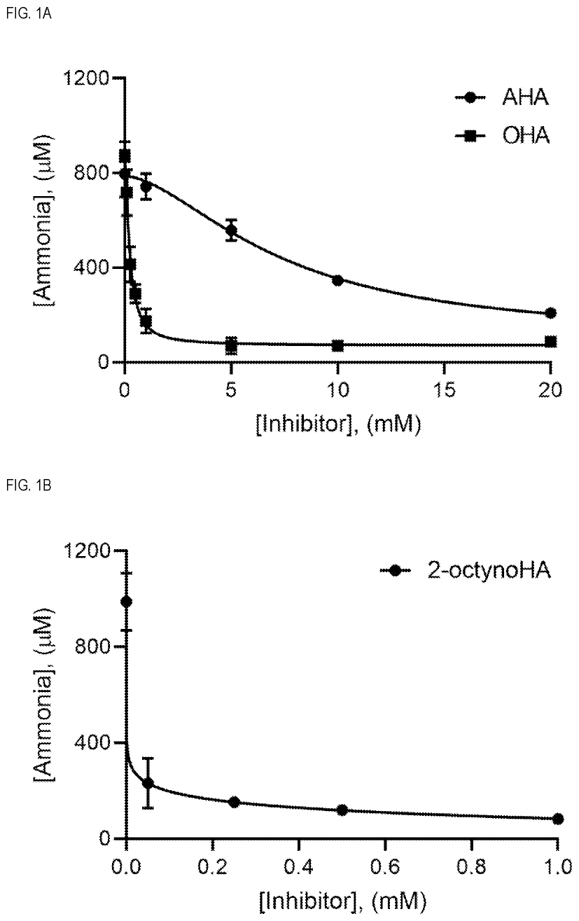

Results show that 2-octynoHA exhibits stronger bacterial urease inhibitory activity than AHA and OHA. The IC₅₀ for 2-octynoHA was approximately 0.038 mM compared to 0.23 mM and 7.3 mM for OHA and AHA, respectively. (FIGS. 1A-B).

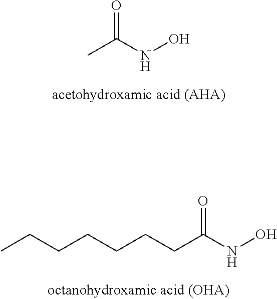

### Example 7: In Vivo Efficacy of 2-Octynohydroxamic Acid in Bile-Duct Ligated Rats

This in vivo study was conducted by Amplia PharmaTek Inc. in Montreal, Canada.

Efficacy of 2-octynoHA was evaluated in the bile-duct ligated (BDL) rat model, which is recommended by the International Society for Hepatic Encephalopathy and Nitrogen Metabolism (ISHEN) as an animal model of hepatic encephalopathy (HE) associated with chronic liver cirrhosis. This model is obtained by obstruction of the common bile duct that causes impairment of liver function and thereby development of hyperammonemia and low-grade HE.

In this study, on the DO blood ammonia levels were measured (n=4, 27.25±6.18 μmol/L) using PocketChem™ (Arkray, Japan) after which all rats were subjected to the BDL surgery. Right before surgery all animals were given injection of slow-release buprenorphine (lasting 72 h) between the shoulder blades. Then, animals were anesthetized with isoflurane in O₂. Depth of anesthesia was checked by toe-pinching. Further, a midline incision was made (2 cm), and then the common bile duct was isolated of surrounding tissue, tightly ligated twice with silk suture and cut between the 2 ligations. Abdominal incision was closed with Vicryl™ 4-0 for muscle layers; skin was closed with staples and one drop of Vetbond™ tissue glue was applied to secure closing. Animals were then placed on heating pad for recovery. For the first 4 weeks post-surgery, animals were monitored for pain level, extent of jaundice, body weight (BW) and disease scores.

Twenty-eight days after the surgery 4 male CD® rats (Charles River Laboratories, Canada) received twice a day 30 mg/kg of 2-octynoHA dissolved in a HPβCD solution (1:1 molar ratio of 2-octynoHA to HPβCD). The treatment was administered via gavage at 4 mL/kg.

Three days after initiating the treatment with 2-octynoHA the mean ammonia levels dropped from 53±30.31 μmol/L to 11±5.68 μmol/L.

### Example 8: Cytotoxicity Evaluation of Hydroxamic Acids in Caco-2 Cells

The compounds used were 2-octynoHA, OHA (Tokyo Chemical Industry Co., Ltd.; Japan) and AHA (Sigma-Aldrich, St. Louis, MO) The human colorectal adenocarcinoma cell line Caco-2 was obtained from American Type Culture Collection. The cells were cultured in complete medium containing DMEM (Dulbecco's modified Eagle's medium) high glucose, GlutaMax™, pyruvate (Thermo Fisher Scientific, Waltham, MA) supplemented with 10% fetal bovine serum (Thermo Fisher Scientific, Waltham, MA), 1% penicillin-streptomycin (Thermo Fisher Scientific, Waltham, MA) and 15 mM HEPES (Thermo Fisher Scientific, Waltham, MA). The cells were incubated at 37° C. in a humidified atmosphere with 5% CO₂. Caco-2 cells were used in the experiments from passage 50 to 60 and were tested negative for mycoplasma.

The cytotoxicity of HAs was assessed using Cell Counting Kit-8 (CCK-8, Sigma-Aldrich, St. Louis, MO), a colorimetric assay based on tetrazolium salt WST-8 which is reduced by dehydrogenases in cells to an orange-colored formazan. The absorbance of formazan at 450 nm is directly proportional to the number of living cells.

In order to test cytotoxicity of HAs, Caco-2 cells were seeded in a 96-well plate at a density of 5×10³ cell/well and grown for 1 day. Then, cells were treated with an increasing range of inhibitors' concentrations and incubated for another 24 h at 37° C., 5% CO₂. As positive and negative controls, complete medium with 10 mM hydrogen peroxide and complete medium only were used, respectively. After incubation, cells were washed with phosphate buffered saline (Thermo Fisher Scientific, Waltham, MA), treated with DMEM without phenol red (Thermo Fisher Scientific, Waltham, MA) containing CCK-8 reagent and incubated for another 2 h at 37° C. in a humidified atmosphere with 5% CO₂. The absorbance of medium at 450 nm was measured using Tecan Infinite M200 PRO plate reader. Caco-2 cell viability was calculated as a percentage of the medium control.

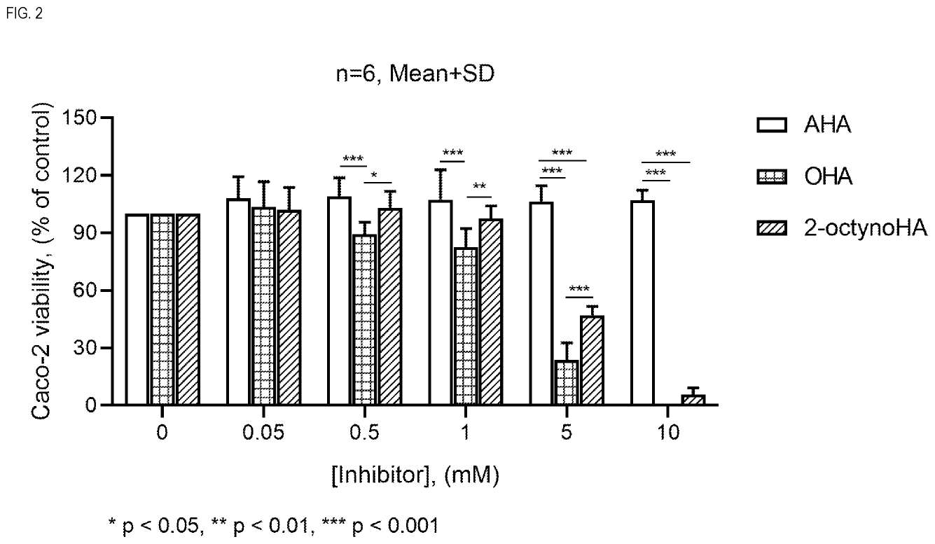

As shown in FIG. 2, 2-octynoHA was not cytotoxic up to 1 mM, but at higher concentrations it had cytotoxic effects decreasing cell viability down to ca. 6% at 10 mM concentration. OHA expressed stronger cytotoxic effects compared to 2-octynoHA, at 10 mM no live cells were observed. As for AHA, no cytotoxicity was detected up to 10 mM. Cell viability in the presence of hydrogen peroxide 10 mM was 1.79±6%.

### Example 9: Mutagenicity Evaluation of Hydroxamic Acids

The compounds used were 2-octynoHA, OHA (Tokyo Chemical Industry Co., Ltd.; Japan) and AHA (Sigma-Aldrich Co., St. Louis, MO) The mutagenic potential of hydroxamic acids was assessed using the standard Ames test which determines the ability of the test compounds to induce reverse mutations in various bacterial strains. A commercial microplate format test kit (Ames MPF™ Penta 1, Xenometrix, Switzerland) was used for this experiment. The most commonly used strains in the Ames tests, S. typhimurium with point mutations in the histidine operon and E. coli with mutations in the tryptophan operon were utilized as the model systems. The mutagenicity of the three hydroxamic acids was investigated in four S. typhimurium strains (TA98, TA100, TA1535, TA1537) and in the mixture of two E. coli strains (wp2 [pKM101] and wp2 uvrA) with and without metabolic activation (S9 fraction) according to the manufacturer's protocol.

Bacteria were exposed to 6 concentrations of the test compounds, solvent control (DMSO) and positive control substances (concentrations listed in Tables I-II below) for 90 min in the medium containing enough histidine for S. typhimurium or tryptophan for E. coli to support cell growth. After that, the cultures media were diluted with pH indicator solution lacking histidine or tryptophan and aliquoted into 48 wells of a 384-well plate. Within 48 hours, media containing cells that reverted to amino acid prototrophy turned yellow as bacteria metabolism reduced the pH of the media. The number of yellow wells containing revertant colonies was counted for each dose of the test compounds and compared to the solvent control.

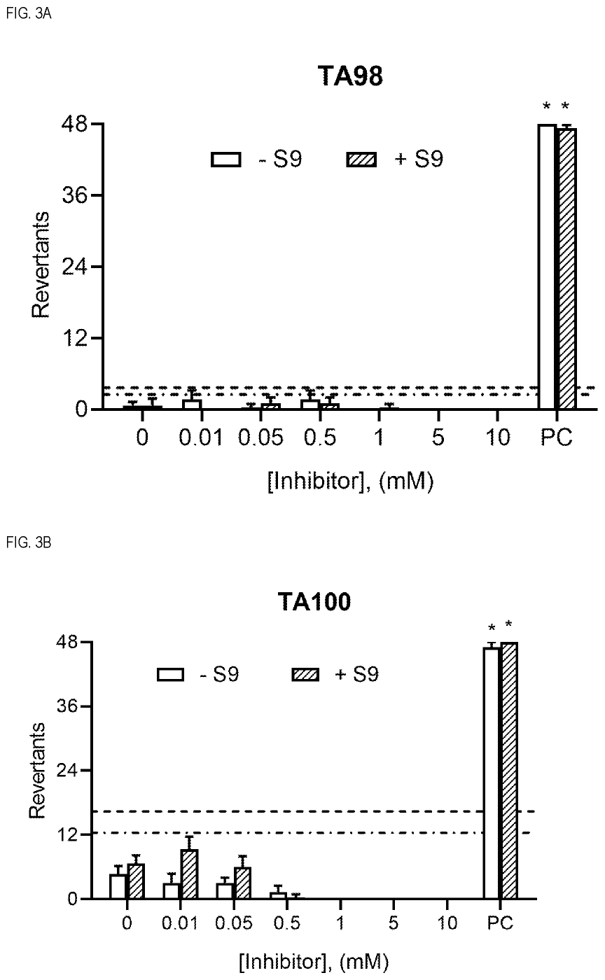

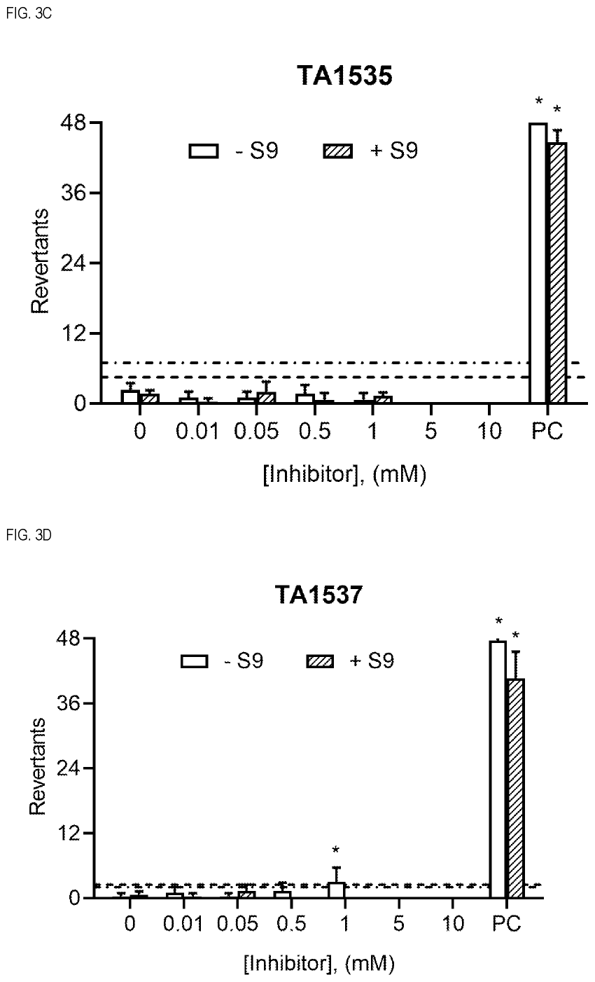

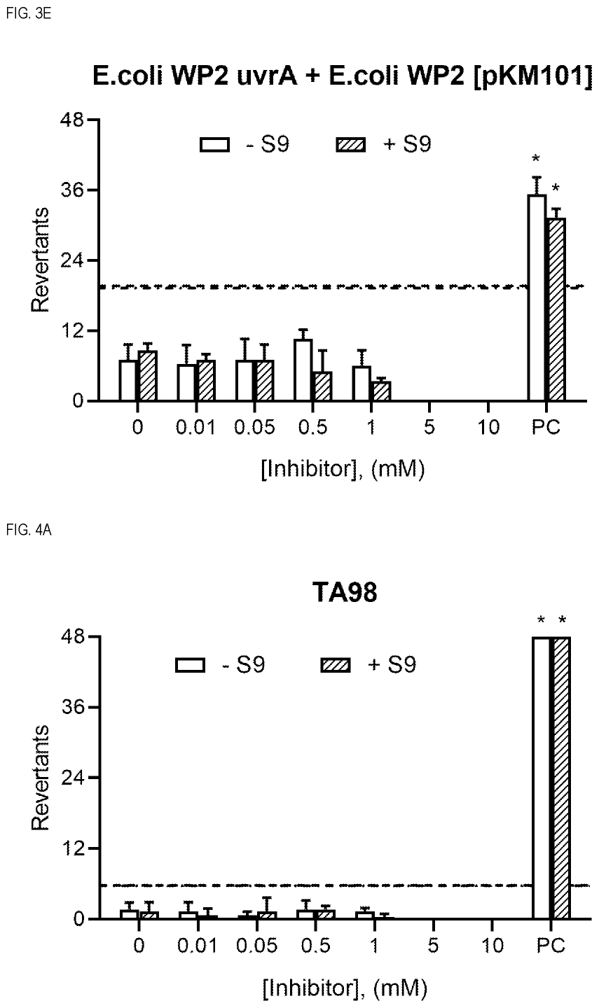

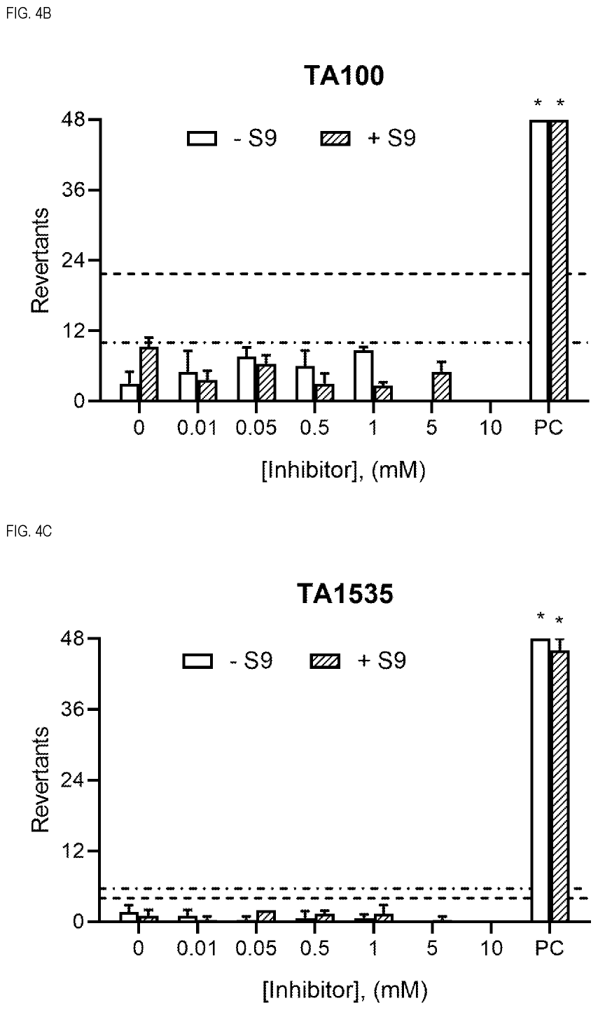

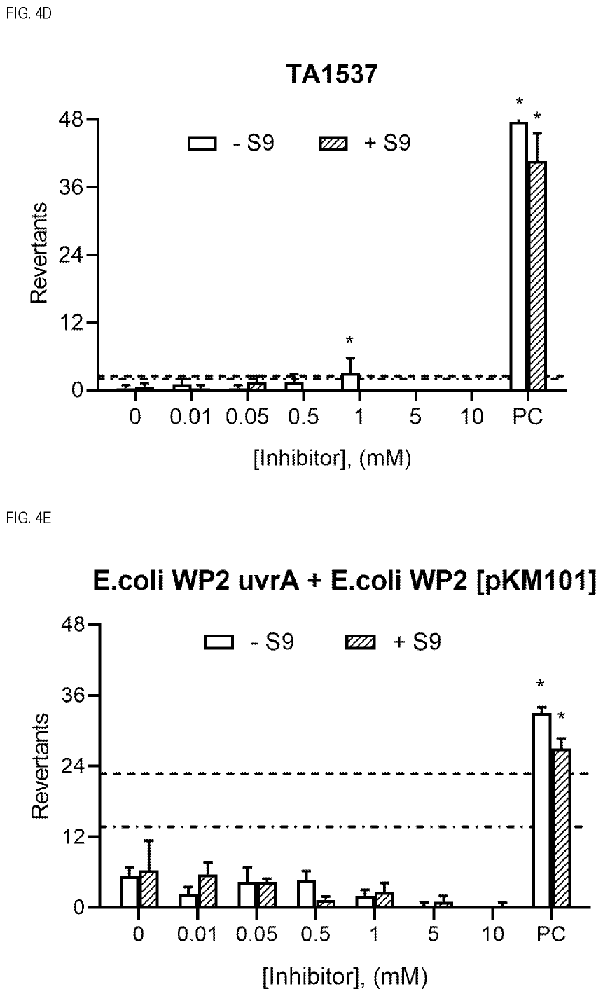

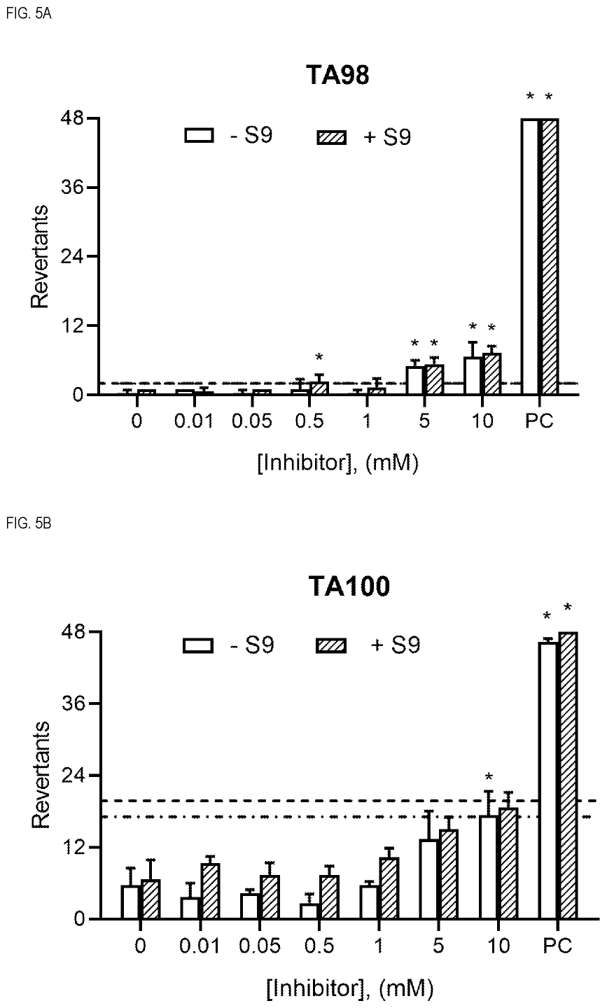

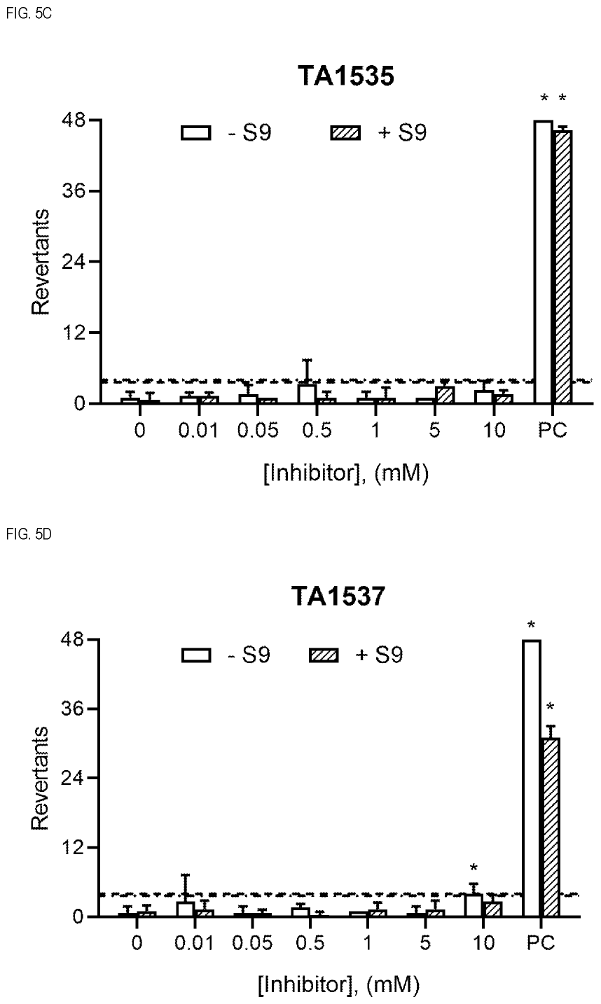

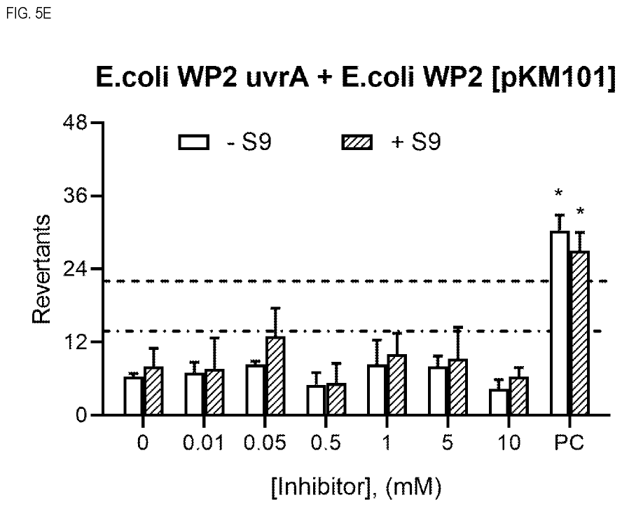

As shown in FIGS. 3A-E, 2-octynoHA is not mutagenic up to 1 mM in any of the strains with or without metabolic activation. Similar results were observed for OHA (FIG. 4A-E) which was shown to be not mutagenic in any of the tested strains. As for AHA, mutagenic potential was detected in TA98, TA100 and TA1537 strains at 5 and 10 mM (FIG. 5A-E). These data suggest that all compound display low mutagenic potential.

TABLE I
Positive controls for the assay without S9 fraction.

| | | Final |
|---|---|---|
| | | concentration |
| Strain | Chemical | (μg/mL) |
| TA98 | 2-Nitrofluorene | 2 |
| TA100 | 4-Nitroquinoline-N-oxide | 0.1 |
| TA1535 | N⁴-Aminocytidine | 100 |
| TA1537 | 9-Aminoacridine | 15 |
| E. coli wp2 uvrA + E. coli | 4-Nitroquinoline-N-oxide | 2 |
| wp2 [pKM101] | | |

TABLE II
Positive controls for the assay with S9 fraction.

| | | Final |
|---|---|---|
| | | concentration |
| Strain | Chemical | (μg/mL) |
| TA98 | 2-Aminoanthracene | 0.5 |
| TA100 | 2-Aminoanthracene | 1.25 |
| TA1535 | 2-Aminoanthracene | 2.5 |
| TA1537 | 2-Aminoanthracene | 2.5 |
| E. coli wp2 uvrA + E. coli | 2-Aminofluorene | 400 |
| wp2 [pKM101] | | |

### Example 10: Activity of Unsaturated Alkylated Hydroxamic Acids in the Rat Caecum Content Assay

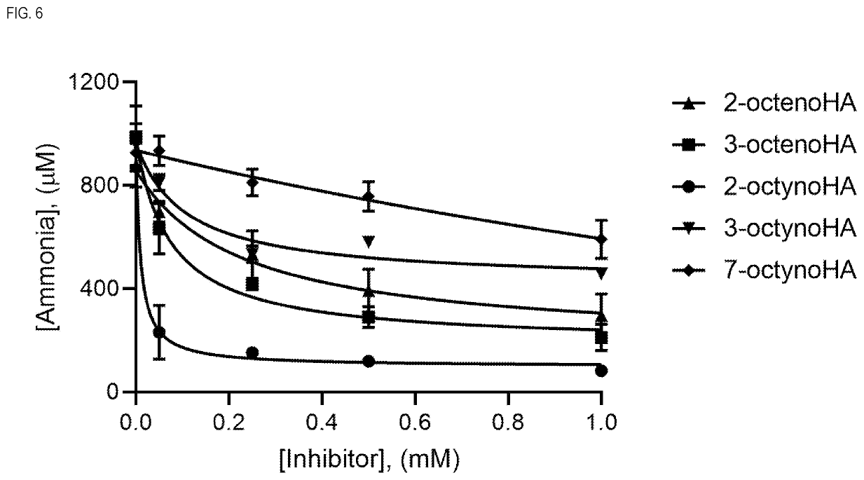

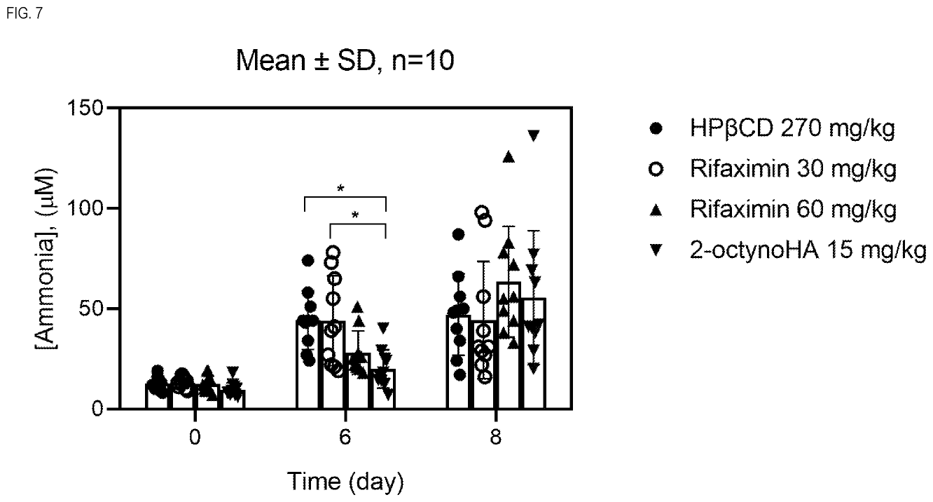

Besides 2-octynoHA, the urease inhibitory activity of other unsaturated alkylated HAs consisting of 8 carbons was assessed. These included 2-octenohydroxamic acid (2-octenoHA), 3-octenohydroxamic acid (3-octenoHA), 3-octynohydroxamic acid (3-octynoHA) and 7-octynohydroxamic acid (7-octynoHA). Prior to assessing potency in the rat caecum content assay, stock solutions of 2-octenoHA and 3-octenoHA were prepared using HPβCD solution (1:4 molar ratio of HA to HPβCD), stock solutions of 3-octynoHA and 7-octynoHA were prepared using mixture of 10% DMSO and HPβCD solution. Results are shown in FIG. 6. The 7-octynoHA presented a lower potency than unsaturated alkylated HAs having a double/triple bond at positions 2 or 3.

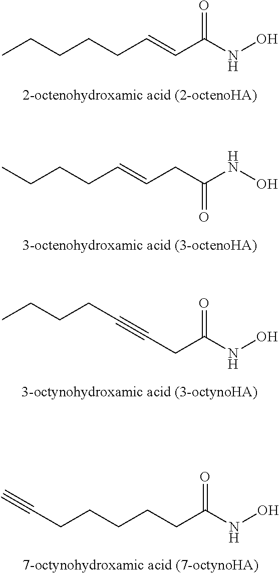

### Example 11 In Vivo Efficacy of 2-Octynohydroxamic Acid in N-Nitrosodiethylamine Induced Liver Disease in Rats

The in vivo study was conducted by Wuhan Servicebio Technology Co., in China.

Efficacy of 2-octynoHA was assessed in male Sprague Dawley rats (Beijing Vital River Laboratory Animal Technology Co., Ltd, China) in a model of acute liver disease. The liver damage was induced by reoccurring intraperitoneal injections of N-nitrosodiethylamine (DEN) 60 mg/kg on days 1, 3, 5, and 7.

Four groups of rats were included. The first control group was treated with a solution of HPβCD 270 mg/kg (n=10). The second group was treated with a suspension of rifaximin 30 mg/kg (n=10). The third group was treated with a suspension of rifaximin 60 mg/kg (n=10). The fourth group was treated with 15 mg/kg of 2-octynoHA dissolved in HPβCD solution (1:1 molar ratio of 2-octynoHA to HPβCD) (n=10). All groups received the treatment via gavage twice a day from day 3 to day 8. Blood samples were collected sublingually on days 0, 6 and 8. Blood ammonia level was measured in collected samples using PocketChem™ (Arkray, Japan).

Blood ammonia levels on day 6 in rats treated with 2-octynoHA (20±9.6 μM) were significantly lower compared to the control group treated with HPβCD 270 mg/kg (44.3±14.7 μM) and to the group treated with rifaximin 30 mg/kg (44±22.4 μM).

### Example 12: Stability of 2-octynoHA in Different Conditions

Preliminary stability of 2-octynoHA was assessed in HBSS pH 7.4 supplemented with HEPES 15 mM and compared to stability of the molecule in ultra-pure water.

Both solutions of 2-octynoHA were prepared to a final concentration of 0.5 mg/mL. 2-octynoHA was incubated for ca. 20 min at 37° C. until fully dissolved in the corresponding media. Once solutions were prepared, they were subjected to UHPLC-UV analysis within ca. 1 h while being kept at room temperature. For chromatographic separation and spectrophotometric analysis, 2 μL of each solution were injected. The chromatographic separation was performed on a 10 cm Polar C18 UHPLC column with a mobile phase composed of water, acetonitrile and formic acid 0.1%.

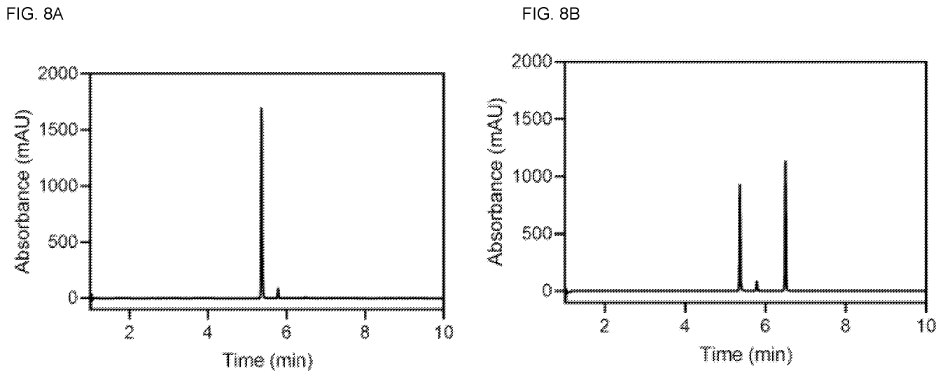

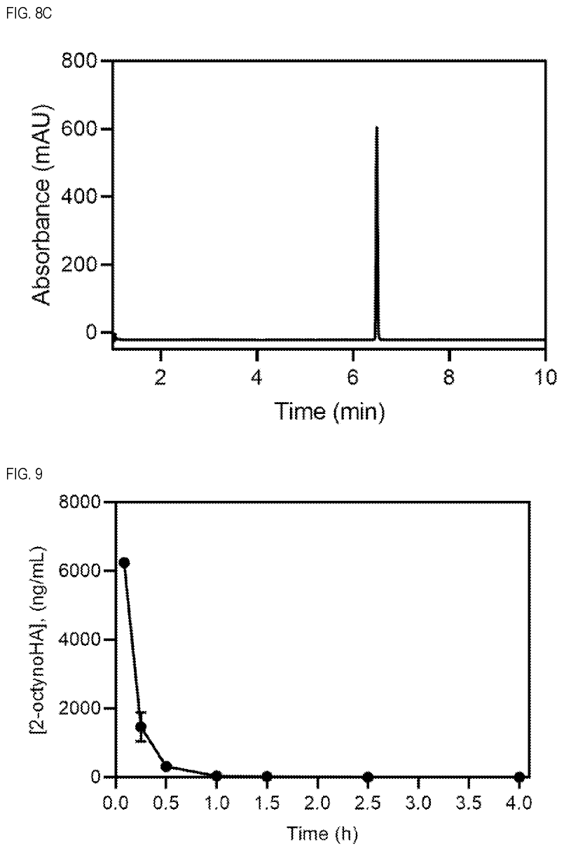

Detection was performed on a diode array detector by monitoring UV absorbance at 200 nm. 2-octynoHA was detected at 200 nm with RT=5.36 min in both solutions. No degradation was noted in a water solution (FIG. 8A), while in a buffer at pH 7.4, 2-octynoHA was degraded to a product eluting at RT=6.49 min (FIG. 8B).

In order to identify the product of degradation, a solution of 2-octynoHA 10 mM was prepared in phosphate buffer (KH₂PO₄ 200 mM pH 6.8) and left to incubate overnight at 37° C. Complete degradation of 2-octynoHA was confirmed by UHPLC-UV analysis (FIG. 8C). The product of degradation was then extracted with chloroform, the organic phase was collected, dried over Na₂SO₄ and filtered. The solvent was evaporated, the obtained substance was dissolved in deuterated dimethyl sulfoxide (DMSO-d6) for ¹H NMR analysis.

¹H NMR (400 MHz, DMSO-d6) δ 10.99 (s, 1H), 5.75 (s, 1H), 2.58 (t, J=7.2 Hz, 2H), 1.61-1.54 (m, 2H), 1.32-1.24 (m, 4H), 0.88-0.85 (t, J=7.2 Hz, 3H).

The identified structure (degradation product of 2-octynoHA) is shown below. These data indicate that 2-octynoHA loses its hydroxamate group when incubated in buffer solution having a near neutral pH.

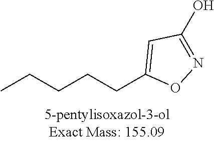

### Example 13: Pharmacokinetics (PK) of 2-octynoHA in Male Beagle Dogs

Three PK studies were conducted in the Institut national de la recherche scientifique (Laval, QC, Canada).

The main objective of the experiment was to quantify the levels of 2-octynoHA in the plasma of male beagle dogs following I.V. administration of a solution of 2-octynoHA with HPβCD (1:1 molar ratio) (PK 1), oral (P.O.) administration of 2-octynoHA formulated in an uncoated gelatin capsule size 0 (PK 2), and oral administration of 2-octynoHA formulated in a coated (Eudragit S100 15% w/w, triethyl citrate 5% w/w, isopropyl alcohol 40% w/w, ethanol 40% w/w) gelatin capsule size 0 for colonic delivery (PK 3) (Table Ill). For each of the studies, three animals were administered with a single dose of the corresponding treatment. Plasma samples were collected at pre-determined time points (Table Ill).

TABLE III
Description of the PK studies

| | | Dose of | | |
|---|---|---|---|---|
| Study | | 2-octynoHA | Route of | Plasma collection time |
| ID | Treatment | per dog | administration | points |
| PK 1 | Sterile solution of 2- | 100 mg | I.V. | Pre-dose, 5 min, 0.25, 0.5, |
| | octynoHA with HPβCD | | | 1, 1.5, 2.5, 4, 6, 8 and 24 h |
| | (1:1 molar ratio) | | | post-dose |
| PK 2 | 2-octynoHA in a gelatin | 300 mg | P.O. | Pre-dose, 0.25, 0.5, 1, 2, 4, |
| | capsule | | | 6, 12 h post-dose |
| PK 3 | 2-octynoHA in a coated | 300 mg | P.O. | Pre-dose, 0.5, 1, 2, 4, 6, 8, |
| | gelatin capsule | | | 12 and 24 h post-dose |

In PK 1, the plasma samples were extracted by protein precipitation prior to analysis. In brief, 40 μL plasma was precipitated with 2 volumes (80 μL) of an organic solvent solution consisting of acetonitrile:methanol (80:20 v/v) containing 1% formic acid. Samples were vortex-mixed and centrifuged at 13,000 rpm. 100 μL of supernatant were further diluted with 100 μL water containing 0.1% formic acid (final dilution factor of 6×). The sample extracts were transferred into a 96-well plate. Four μL plasma extract were injected for chromatographic separation and high resolution/accurate mass analysis. Separation was performed on a 5 cm C18 UPLC column, detection and quantification were performed on a quadrupole time-of-flight (QTof) mass spectrometer in the positive ion mode.

In both PK 2 and PK 3, prior to extraction, plasma samples (ca. 700 μL) were acidified by adding 15 μL of 50% phosphoric acid in water. Then, plasma samples (40 μL) were extracted by protein precipitation by adding 2 volumes (80 μL) of an organic solvent solution consisting of acetonitrile:methanol (80:20 v/v) containing 0.2% formic acid.

Samples were vortex-mixed and centrifuged at 13,000 rpm for 5 min. 100 μL of supernatant were further diluted with 100 μL water containing 0.1% formic acid (final dilution factor of 6×). The sample extracts were transferred into a 96-well plate. Five μL were injected for chromatographic separation and further mass analysis. The chromatographic separation was performed on a 10 cm C18 UPLC column and the MS detection and quantification was performed on a quadrupole time-of-flight (QTof) mass spectrometer in the positive ion mode.

Concentrations of 2-octynoHA was quantified based on the calibration curve. In the PK 1, 2-octynoHA was detected at the m/z 156.1 with the retention time (RT) of 4.12 min. FIG. 9 shows the concentration profile of 2-octynoHA up to 4 h post-dose. Concentration values below the lower limit of quantitation (LLOQ) (i.e., lowest amount of an analyte in a sample that can be quantitatively determined with suitable precision and accuracy) were considered equal to zero.

AUC₀₋ₜ (t=4 h) value was calculated using the non-compartmental analysis after I.V. dosing (PK Solver 2.0), and it was ca. 1781 ng/mL*h.

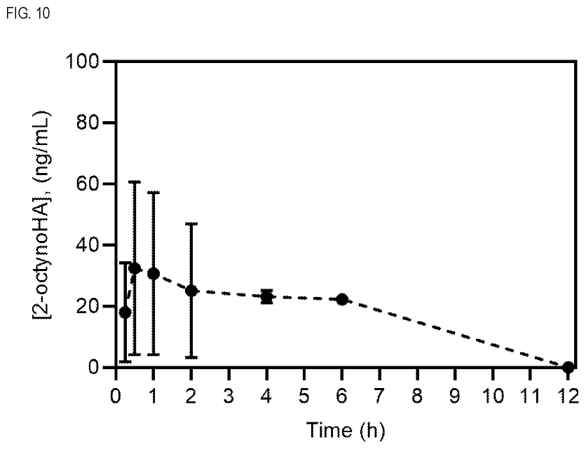

In PK 2, 2-octynoHA was detected at the m/z 156.1 with RT=5.52 min. FIG. 10 shows the concentration profile of 2-octynoHA up to 12 h post-dose. Concentration values below LLOQ were considered equal to zero. The tₘₐₓ was observed at 0.5 h. AUC₀₋ₜ (t=12 h) value was calculated using the non-compartmental analysis after extravascular dosing (PK Solver 2.0), it was ca. 213 ng/mL*h. Based on the dose-adjusted AUC₀₋ₜ for 2-octynoHA concentration profiles in PK 1 and PK 2, bioavailability of 2-octynoHA after oral administration in an uncoated gelatin capsule was ca. 4.5%.

In PK 3, 2-octynoHA could not be quantified in the plasma (below LLOQ) indicating lower systemic exposure than that obtained after administration of the uncoated capsule.

These data suggest that the administration of 2-octynoHA via a colonic capsule reduced systemic exposure which is desired because the main site of action is the colon. The scope of the claims should not be limited by the embodiments set forth in the examples, but should be given the broadest interpretation consistent with the description as a whole.

### REFERENCES

Bosoi C. R. and Rose, C. F. “Identifying the direct effects of ammonia on the brain,” Metab Brain Dis, vol. 24, no. 1, pp. 95-102, March 2009, doi: 10.1007/s11011-008-9112-7.
Braissant, O., McLin, V. A. and Cudalbu, C., “Ammonia toxicity to the brain,” Journal of Inherited Metabolic Disease, vol. 36, no. 4, pp. 595-612, 2013, doi: 10.1007/s10545-012-9546-2.
Gupta, S. P., “Hydroxamic acids: A unique family of chemicals with multiple biological activities”, Springer Science & Business Media, 2013.
Häberle, J., “Clinical and biochemical aspects of primary and secondary hyperammonemic disorders,” Arch. Biochem. Biophys., vol. 536, no. 2, pp. 101-108, August 2013, doi: 10.1016/j.abb.2013.04.009.
Muri E. M. F. and Barros, T. G., “Hydroxamic acids as inhibitors of urease in the treatment of Helicobacter pylori infections,” in Hydroxamic acids: A unique family of chemicals with multiple biological activities, S. P. Gupta, Ed. 2013, pp. 241-253.
Philip et al., “Colon Targeted Drug Delivery Systems: A Review on Primary and Novel Approaches”, Oman Med J. 2010 April; 25(2): 79-87.
Rivera-Flores et al., “Manipulation of microbiota with probiotics as an alternative for treatment of hepatic encephalopathy”, Nutrition. 2020 May; 73: 110693.
Rose C. F. et al., “Hepatic encephalopathy: Novel insights into classification, pathophysiology and therapy,” Journal of Hepatology, vol. 73, no. 6, pp. 1526-1547, December 2020, doi: 10.1016/j.jhep.2020.07.013 (2020a).
Shen S. and Kozikowski A. P., “Why Hydroxamates May Not Be the Best Histone Deacetylase Inhibitors—What Some May Have Forgotten or Would Rather Forget?,” ChemMedChem, vol. 11, no. 1, pp. 15-21, January 2016, doi: 10.1002/cmdc.201500486.
Swaminathan, M., Ellul, M. A. and Cross, T. J., “Hepatic encephalopathy: current challenges and future prospects,” Hepatic Medicine: Evidence and Research, Mar. 22, 2018. https://www.dovepress.com/hepatic-encephalopathy-current-challenges-and-future-prospects-peer-reviewed-article-HMER (accessed Feb. 25, 2021).
Vilstrup H. et al., “Hepatic encephalopathy in chronic liver disease: 2014 Practice Guideline by the American Association for the Study of Liver Diseases and the European Association for the Study of the Liver,” Hepatology, vol. 60, no. 2, pp. 715-735, August 2014, doi: 10.1002/hep.27210.
Walker, V., “Ammonia metabolism and hyperammonemic disorders,” in Advances in Clinical Chemistry, vol. 67, G. S. Makowski, Ed. Elsevier, 2014, pp. 73-150. doi: 10.1016/bs.acc.2014.09.002.

1. An enteral al or urinary pharmaceutical formulation comprising (A) a compound of formula I, 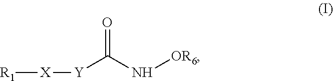
wherein R₁ is a C1-C3 alkyl;
X is —C(R₂)(R₃)—C(R₄)(R₅)— or —C≡C—,
wherein R₂, R₃, R₄, and R₅ are each independently H or methyl;
Y is —CH═CH—CH₂—, —CH₂—CH═CH—; —C≡C—CH₂; —CH₂—C≡C—; or —C(R₇)(R₈)—C(R₉)(R₁₀)—C(R₁₁)(R₁₂)—,
wherein R₇, R₈, R₉, R₁₀, R₁₁ and R₁₂, are each independently H or methyl;
provided that (1) when X is —C≡C—, Y is —C(R₇)(R₈)—C(R₉)(R₁₀)—C(R₁₁)(R₁₂)— and that (2) when Y is not —C(R₇)(R₈)—C(R₉)(R₁₀)—C(R₁₁)(R₁₂)—, X is —C(R₂)(R₃)—C(R₄)(R₅)—; and
R₆ is H or CH₃,
or a stereoisomer or a mixture thereof, or a pharmaceutically acceptable salt, ester or solvate thereof; and
(B) (a) at least one pharmaceutically acceptable excipient; (b) at least one other therapeutic agent; or (c) a combination of (a) and (b).

2. The formulation of claim 1, wherein R₁ is ethyl.

3. The formulation of claim 1, wherein
(i) X is —C(R₂)(R₃)—C(R₄)(R₅)—;
(ii) R₁ is ethyl;
(iii) R₂ is H;
(iv) R₃ is H;
(v) R₄ is H;
(vi) R₅ is H;
(vii) R₆ is H;
(viii) Y is —CH═CH—CH₂—, —CH₂—CH═CH—, —C≡C—CH₂—, or —CH₂—C≡C—; or
(ix) a combination of at least two of (i) to (viii).

4. The formulation of claim 1, wherein
(i) X is —C(R₂)(R₃)—C(R₄)(R₅)—;
(ii) R₁ is ethyl;
(iii) R₂ is H;
(iv) R₃ is H;
(v) R₄ is H;
(vi) R₅ is H;
(vii) R₆ is H; and
(viii) Y is —CH═CH—CH₂—, —CH₂—CH═CH—, —C≡C—CH₂—, or —CH₂—C≡C—.

5. The formulation of claim 1, wherein the compound is of formula Ia: 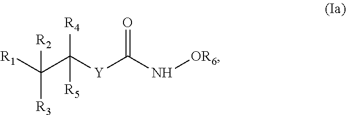
wherein R₁, R₂, R₃, R₄, R₅ and R₆ are as defined in claim 1 and Y is CH═CH—CH₂—, —CH₂—CH═CH—, —C≡C—CH₂—, or —CH₂—C≡C—, or a stereoisomer or a mixture thereof, or a pharmaceutically acceptable salt, ester or solvate thereof.

6. The formulation of claim 1, wherein Y is CH═CH—CH₂—, —CH₂—CH═CH— or —CH₂—C≡C—.

7. The formulation of claim 1, wherein Y is —CH₂—C≡C—.

8. The formulation of claim 1, wherein the compound is: 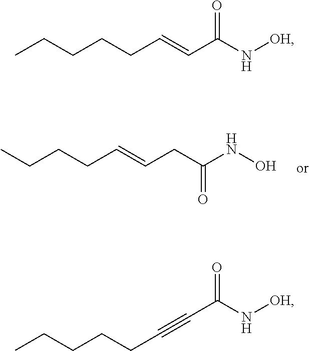
or a stereoisomer or a mixture thereof, or a pharmaceutically acceptable salt, ester or solvate thereof.

9. The formulation of claim 1, wherein the compound is 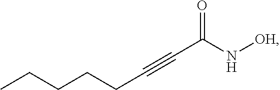
or a stereoisomer or a mixture thereof, or a pharmaceutically acceptable salt, ester or solvate thereof.

10. The formulation of claim 1, for enteral or urinary administration.

11. The formulation of claim 10, for delivery in the ileum or colon.

12. The formulation of claim 1 comprising an enteric coating.

13. A method of treating or preventing an ammonia-associated disease or disorder, or a symptom thereof in a subject in need thereof, comprising administering a therapeutically effective amount of the compound, stereoisomer, mixture, salt, ester, solvate or formulation of claim 1 to the subject.

14. The method of claim 13, wherein the ammonia-associated disease or disorder is hyperammonemia.

15. The method of claim 13, wherein the compound, stereoisomer, mixture, salt, ester, solvate or formulation is administered enterally or through the urinary tract.

16. The formulation of claim 1, which is an enteral formulation.

17. The formulation of claim 1, wherein Y is CH═CH—CH₂—, or —CH₂—C≡C—.

18. The formulation of claim 1, for enteral administration.

19. The formulation of claim 1, for enteral administration through a delayed release formulation.

20. The method of claim 13, wherein the subject in need thereof has hepatic encephalopathy or a urea cycle disorder.

21. The method of claim 13, wherein the compound, stereoisomer, mixture, salt, ester, solvate or formulation is administered enterally.

22. The method of claim 13, wherein the compound, stereoisomer, mixture, salt, ester, solvate or formulation is administered to the ileum or the colon.
